(19) United States
(12) Patent Application Publication
(10) Pub. No.: US 2008/0248089 A1
(43) Pub. Date: Oct. 9, 2008

(54) EDIBLE PRODUCTS FOR THE TREATMENT OF NASAL PASSAGES

(75) Inventors: Diane Bugge, Rockaway, NJ (US); Joan E. Harvey, Morgantown, PA (US); Susan Pettigrew, Suffern, NY (US)

Correspondence Address: HOFFMANN & BARON, LLP, 6900 JERICHO TURNPIKE, SYOSSET, NY, 11791, US

(73) Assignee: CADBURY ADAMS USA LLC, Parsippany, NJ (US)

(21) Appl. No.: 12/100,046

(22) Filed: Apr. 9, 2008

Related U.S. Application Data
(60) Provisional application No. 60/922,444, filed on Apr. 9, 2007.

(51) Int. Cl. A61K 9/00; A61K 31/26; A61K 9/68

(52) U.S. Cl. 424/440; 514/514; 424/439; 424/48

(57) ABSTRACT

The present invention relates to oral delivery systems, such as confectionery and chewing gum compositions, and methods for imparting the perception of nasal clearing to a user. In particular, the present invention includes confectionery and chewing gum compositions including a compound selected from 4-pentenyl isothiocyanate, 5-hexenyl isothiocyanate, 3-butenyl isothiocyanate, 3-methylthiopropyl isothiocyanate, and any combinations thereof and methods. The delivery systems promote the perception of nasal clearing in the user.

### CROSS REFERENCE TO RELATED APPLICATION

This non-provisional application claims priority from U.S. Provisional Patent Application No. 60/922,444, filed Apr. 9, 2007, the entirety of which is incorporated by reference herein.

### FIELD

The present invention relates to edible products containing a compound selected from 4-pentenyl isothiocyanate, 5-hexenyl isothiocyanate, 3-butenyl isothiocyanate, 3-methylthiopropyl isothiocyanate, and combinations thereof.

### BACKGROUND

Confectioneries available today generally include sweeteners, natural or artificial flavors, and a variety of additional components tailored to provide specific characteristics. Chewing gums available today generally contain a water-insoluble gum base, sweeteners, natural or artificial flavors, and a variety of additional components tailored to provide specific characteristics. For example, some chewing gums may include plasticizers or softeners to improve consistency during chew. Other confectioneries and chewing gums, for instance, may include physiological cooling agents to provide a cooling sensation upon consumption by the user. Oral delivery of actives, such as flavors, sweeteners, sensates and therapeutic agents, for their intended purpose, is one of the main objectives of confectionery and chewing gum compositions.

It is known, as described in U.S. Patent Application No. 2003/0170322, to use isothiocyanates, specifically allyl isothiocyanates, in oral care agents for their antibacterial activities and promotion of saliva secretion. However, as described in U.S. Patent Application No. 2003/0064131, it is well known that allyl isothiocyanate has a distinctly pungent mustard-like flavor. Although allyl isothiocyanate has been used in oral compositions, its irritating mustard-like flavor makes use of the ingredient particularly undesirable for incorporation in a confectionery or chewing gum composition.

It has been found that isothiocyanates may also impart the perception of nasal clearing. It would be desirable to create a chewing gum or confectionery which includes an isothiocyanate and would provide a user with the perception of nasal clearing without imparting any undesirable taste or flavor thereto, such as mustard or onion-like flavors. It has been found that 4-pentenyl isothiocyanate, 5-hexenyl isothiocyanate, 3-butenyl isothiocyanate, and 3-methylthiopropyl isothiocyanate, provide the aroma advantages of nasal clearing, without imparting undesirable taste or flavor to oral compositions into which such compounds are incorporated.

There is a need, therefore, for new chewing gum compositions and confectioneries, which provide the desired advantage of imparting the perception of nasal clearing to a user with the incorporation of a compound selected from 4-pentenyl isothiocyanate, 5-hexenyl isothiocyanate, 3-butenyl isothiocyanate, 3-methylthiopropyl isothiocyanate, and combinations thereof without imparting any undesirable taste or flavor.

### SUMMARY

In some embodiments, there is an oral composition including a wasabi extract including a compound selected from 4-pentenyl isothiocyanate, 5-hexenyl isothiocyanate, 3-butenyl isothiocyanate, 3-methylthiopropyl isothiocyanate, and combinations thereof.

In some embodiments there is a confectionery composition including a confectionery carrier and a compound selected from substituted or unsubstituted C₄₋₁₀ alkenyl isothiocyanates.

In some embodiments there is a confectionery composition including a confectionery carrier and a compound selected from substituted or unsubstituted C₄₋₁₀ alkenyl isothiocyanates, 3-methylthiopropyl isothiocyanate, and combinations thereof, and at least one sensate.

In some embodiments there is a confectionery composition including a confectionery carrier and a compound selected from 4-pentenyl isothiocyanate, 5-hexenyl isothiocyanate, 3-butenyl isothiocyanate, 3-methylthiopropyl isothiocyanate, and combinations thereof; and at least one sensate.

In some embodiments there is a composition including a confectionery carrier, a compound selected from substituted or unsubstituted C₄₋₁₀ alkenyl isothiocyanates, 3-methylthiopropyl isothiocyanate, and combinations thereof, and at least one sensate.

In some embodiments there is a composition including a compound selected from substituted or unsubstituted C₄₋₁₀ alkenyl isothiocyanates, 3-methylthiopropyl isothiocyanate, and combinations thereof, and at least one sensate.

In some embodiments there is a confectionery composition including a confectionery carrier, a compound selected from 4-pentenyl isothiocyanate, 5-hexenyl isothiocyanate, 3-butenyl isothiocyanate, 3-methylthiopropyl isothiocyanate, and combinations thereof, and at least one sensate.

In some embodiments there is a composition including a compound selected from 4-pentenyl isothiocyanate, 5-hexenyl isothiocyanate, 3-butenyl isothiocyanate, 3-methylthiopropyl isothiocyanate, and combinations thereof, and at least one sensate.

In some embodiments, there is a multi-region composition including a first region, a second confectionery shell region surrounding the first region and a compound selected from substituted or unsubstituted C₄₋₁₀ alkenyl isothiocyanates, 3-methylthiopropyl isothiocyanate, and combinations thereof.

In some embodiments, there is a multi-region composition including a first region, a second confectionery shell region surrounding the first region and a compound selected from 4-pentenyl isothiocyanate, 5-hexenyl isothiocyanate, 3-butenyl isothiocyanate, 3-methylthiopropyl isothiocyanate, and combinations thereof.

In some embodiments, there is a multi-region composition including a first region and a second confectionery shell region surrounding the first region, the second confectionery shell region including a confectionery carrier, where one of the regions includes a compound selected from substituted or unsubstituted C₄₋₁₀ alkenyl isothiocyanates, 3-methylthiopropyl isothiocyanate, and combinations thereof and the other region includes at least one sensate.

In some embodiments, there is a composition including: a confectionery carrier; a compound selected from substituted or unsubstituted C₄₋₁₀ alkenyl isothiocyanates, 3-methylthiopropyl isothiocyanate, and combinations thereof; and at least one potentiator.

In some embodiments, there is a composition including: a confectionery carrier; a compound selected from 4-pentenyl isothiocyanate, 5-hexenyl isothiocyanate, 3-butenyl isothiocyanate, and combinations thereof; and at least one potentiator.

In some embodiments, there is a composition including: a compound selected from substituted or unsubstituted C₄₋₁₀ alkenyl isothiocyanates, 3-methylthiopropyl isothiocyanate, and combinations thereof; and at least one potentiator.

In some embodiments, there is provided a composition including: a compound selected from 4-pentenyl isothiocyanate, 5-hexenyl isothiocyanate, 3-butenyl isothiocyanate, 3-methylthiopropyl isothiocyanate, and combinations thereof; and at least one potentiator.

In some embodiments, there is a confectionery composition including: a confectionery carrier; at least one compound selected from 4-pentenyl isothiocyanate, 5-hexenyl isothiocyanate, 3-butenyl isothiocyanate, 3-methylthiopropyl isothiocyanate, and combinations thereof; and at least one potentiator.

In some embodiments, there is provided a method of preparing a confectionery product including the steps of (a) providing at least one sweetener; (b) adding a compound selected from 4-pentenyl isothiocyanate, 5-hexenyl isothiocyanate, 3-butenyl isothiocyanate, 3-methylthiopropyl isothiocyanate, and combinations thereof to the sweetener; and (c) forming a confectionery product from the combination of (a) and (b).

In some embodiments, there is a method of preparing a chewing gum product including the steps of (a) providing a gum base; (b) adding a compound selected from a compound selected from 4-pentenyl isothiocyanate, 5-hexenyl isothiocyanate, 3-butenyl isothiocyanate, 3-methylthiopropyl isothiocyanate, and combinations thereof to the elastomer; and (c) forming a chewing gum product from the combination of (a) and (b).

In some embodiments, there is a method of preparing a multi-region confectionery composition including the steps of:
(a) extruding a multi-region rope of a confectionery layer, where the rope includes:
   (i) a first region composition;
   (ii) a second confectionery shell region surrounding the first region composition; and
   (iii) a compound selected from 4-pentenyl isothiocyanate, 5-hexenyl isothiocyanate, 3-butenyl isothiocyanate, 3-methylthiopropyl isothiocyanate, and combinations thereof, where the compound is located in one or more of the first region composition and the second confectionery shell region;
(b) sizing the rope;
(c) feeding the rope into a tablet-forming mechanism; and
(d) forming individual pieces of the center-filled rope.

In some embodiments, there is a method of imparting the perception of nasal clearing to a user including the steps of:
(a) providing an oral delivery system including a chewing gum composition or a confectionery composition including a compound selected from 4-pentenyl isothiocyanate, 5-hexenyl isothiocyanate, 3-butenyl isothiocyanate, 3-methylthiopropyl isothiocyanate, and combinations thereof; and
(b) orally administering the oral delivery system for a time sufficient to produce a nasal clearing perception.

In some embodiments, there is a method of preparing a flavor composition including:
(a) combining at least one flavor compound with a compound selected from 4-pentenyl isothiocyanate, 5-hexenyl isothiocyanate, 3-butenyl isothiocyanate, 3-methylthiopropyl isothiocyanate, and combinations thereof; and
(b) mixing until homogeneous.

### DETAILED DESCRIPTION

The present invention relates to confectionery compositions including chewing gums that provide the advantage of incorporating a compound selected from 4-pentenyl isothiocyanate, 5-hexenyl isothiocyanate, 3-butenyl isothiocyanate, 3-methylthiopropyl isothiocyanate, and combinations thereof, to impart the perception of nasal clearing to a user. Adding the compound to a chewing gum or confectionery composition provides the benefit of imparting the perception of nasal clearing without imparting any undesirable taste or flavor.

As used herein the transitional term “comprising,” (also “comprises,” and the like) which is synonymous with “including,” “containing,” or “characterized by,” is inclusive or open-ended and does not exclude additional, unrecited elements or method steps, regardless of its use in the preamble or the body of a claim.

As used herein, the terms “bubble gum” and “chewing gum” are used interchangeably and are both meant to include any gum compositions.

As used herein, the term “first region” or “center-fill” refers to the innermost region of a multi-region chewing gum or confectionery product. The term “center-fill” does not imply symmetry of a chewing gum or confectionery product, only that the “center-fill” is within another region of the product. In some embodiments, more than one center-fill may be present.

As used herein, the term “chewing gum region” or “confectionery region” or “second confectionery shell region” refers to a region of a center-fill chewing gum or confectionery product, respectively, that may be adjacent to or at least partially surrounding the center-fill, or innermost, region. In some embodiments, the chewing gum region or confectionery region is an intermediate region.

As used herein, the terms “coating” or “coating region” or “third region” are used to refer to the outermost region of a center-fill chewing gum or confectionery product. In some embodiments, the coating may be amorphous or crystalline and it may be continuous or particulate. In some embodiments, the coating or third region may at least partially surround the confectionery shell region. Particulate coatings may be referred to as sanding compositions or dusting compositions. Confections with such particulate coatings may be referred to as sanded or dusted.

As used herein, the terms “surround,” “surrounding,” and the like are not limited to encircling. These terms may refer to enclosing or confining on all sides, encircling or enveloping, and are not limited to symmetrical or identical thicknesses for a region in a multi-region chewing gum or confectionery product.

As used herein, the term “multi-region” confectionery refers to confectionery product formats such as, but not limited to, center-filled (solid, liquid, gas, semi-solid, slurry, and the like, fillings), layered (vertically, horizontally, concentrically, and the like and combinations thereof, with two or more layers), coated, single surface treated (decorated, bottomed, topped, and the like and combinations thereof), multiple surface treated (sprayed, printed, and the like and combinations thereof).

As used herein, the term “confectionery carrier” refers to any confectionery composition to which isothiocyanates have been added. The confectionery carrier can be selected from the group consisting of hard candy, soft candy, chewy candy, center-fill candy, multi-layer candy, cotton candy, pressed tablets, multi-layer pressed tablets, lozenges, edible films, nougats, caramels, frappes, granules, gummies, jellies, taffies, toffees, and chewing gum. Additionally, in embodiments where chewing gum is the confectionery carrier, the chewing gum can be selected from the group consisting of slab, pellet, sticks, balls, cubes, center-fill chewing gums, candy chewing gums, multi-layer chewing gums, deposited chewing gums and compressed chewing gums.

Suitable isothiocyanates useful in some embodiments may include substituted or unsubstituted C₄₋₁₀ alkenyl isothiocyanates, 3-methylthiopropyl isothiocyanate, and combinations thereof. In some embodiments, suitable isothiocyanates may include a compound selected from, 4-pentenyl isothiocyanate, 5-hexenyl isothiocyanate, 3-butenyl isothiocyanate, 3-methylthiopropyl isothiocyanate, and combinations thereof.

More specifically, 4-pentenyl isothiocyanate is represented as:

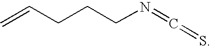

5-hexenyl isothiocyanate is represented as:

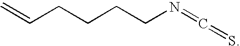

3-butenyl isothiocyanate is represented as:

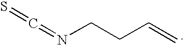

3-methylthiopropyl isothiocyanate is represented as:

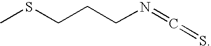

As described herein, a variety of different edible products, such as confectionery compositions including chewing gum compositions, can include one or more of these isothiocyanates to provide products which incorporate the benefit of imparting the perception of nasal clearing without imparting any undesirable taste or flavor. In some embodiments, the one or more isothiocyanates may be combined with one or more sensates such as, but not limited to, cooling agents, warming agents, tingling agents, effervescent agents, and combinations thereof. In some embodiments, the one or more isothiocyanate may be combined with a non-menthol sensate. In other embodiments, the one or more isothiocyanates may be combined with one or more potentiators.

In some embodiments, the time sufficient to produce the nasal clearing perception may be from about 5 seconds to about 60 seconds. In other embodiments, the oral delivery system may further include a long-lasting component, where the time sufficient to produce a nasal clearing perception may be up to about 60 minutes. In other embodiments, the oral delivery system may further include an after-effect component, where nasal passages would feel clear for up to 60 minutes after consumption/expectoration of the oral delivery system.

### Confectionery Compositions

Some embodiments described herein provide confectionery compositions that include confections other than chewing gum compositions. The confectionery compositions may include a confectionery carrier, such as sugar and/or sugarless sweeteners, and a compound selected from 4-pentenyl isothiocyanate, 5-hexenyl isothiocyanate, 3-butenyl isothiocyanate, 3-methylthiopropyl isothiocyanate, and combinations thereof. The isothiocyanates may be present in amounts of about 0.001% to about 3.0% by weight of a non-chewing gum confectionery composition. The confectionery compositions promote the perception of nasal clearing in the user.

Confectionery compositions may be provided in a variety of different forms, such as, for example, hard candy, soft candy, center-fill candy, multi-layer candy, cotton candy, pressed tablets, multi-layer pressed tablets, lozenges, edible films, nougats, caramels, frappes, granules, gummies, jellies, and taffies. The confectionery compositions may also include at least one flavor and a variety of optional additives.

In general, a hard boiled candy confection has a base composed of a mixture of sugar and/or sugarless sweetening agents and other carbohydrate bulking agents kept in an amorphous or glassy condition. In some embodiments, the sweetener itself may act as the carrier for the confectionery composition, or additional carrier components may be employed. Any of the sweeteners as discussed below in the section entitled “Sweetening Ingredients” may be used. A general discussion of the composition and preparation of hard confections may be found in E. B. Jackson, Ed. “Sugar Confectionery Manufacture”, 2nd edition, Blackie Academic & Professional Press, Glasgow UK, (1990), at pages 129-169, as well as in H. A. Lieberman, Pharmaceutical Dosage Forms: Tablets, Volume 1 (1980), Marcel Dekker, Inc., New York, N.Y. at pages 339 to 469, which disclosure is incorporated herein by reference.

Such confectionery compositions may be routinely prepared by conventional methods such as those involving fire cookers, vacuum cookers, and scraped-surface cookers also referred to as high speed atmospheric cookers.

Fire cookers involve the traditional method of making a candy base. In this method, the desired quantity of carbohydrate bulking agent is dissolved in water by heating the agent in a kettle until the bulking agent dissolves. Additional bulking agent may then be added and cooking continued until a final temperature of 145° C. to 156° C. is achieved. The batch is then cooled and worked as a plastic-like mass to incorporate additives such as flavors, colorants and the like.

A high-speed atmospheric cooker uses a heat-exchanger surface which involves spreading a film of candy on a heat exchange surface and heating the candy to 165° C. to 170° C. in a few minutes. The candy is then rapidly cooled to 100° C. to 120° C. and worked as a plastic-like mass enabling incorporation of the additives, such as flavors, colorants and the like.

In vacuum cookers, the carbohydrate bulking agent is boiled to 125° C. to 132° C., vacuum is applied and additional water is boiled off without extra heating. When cooking is complete, the mass is a semi-solid and has a plastic-like consistency. At this point, flavors, colorants, and other additives are admixed in the mass by routine mechanical mixing operations.

The optimum mixing required to uniformly mix the flavors, colorants and other additives during conventional manufacturing of hard confectionery is determined by the time needed to obtain a uniform distribution of the materials. Normally, mixing times of from 4 to 10 minutes have been found to be acceptable.

Once the candy mass has been properly tempered, it may be cut into workable portions or formed into desired shapes. A variety of forming techniques may be utilized depending upon the shape and size of the final product desired.

Soft candy confectionery compositions includes fondants, caramels toffees, fudge, gummies, jellies, chewy candy, marshmallows and nougats and the like and may also include jams and jellies. The preparation of soft confectionery compositions, such as nougat, involves conventional methods, such as the combination of two primary components, namely (1) a high boiling syrup, and (2) a relatively light textured frappe, generally prepared from egg albumin, gelatin, vegetable proteins, such as soy derived compounds, milk derived compounds such as milk proteins, and mixtures thereof. A general discussion of the composition and preparation of such confections may be found in E. B. Jackson. Ed. “Sugar Confectionery Manufacture”, 2nd edition, Blackie Academic & Professional Press. Glasgow UK (1990), at pages 170-235.

The high boiling syrup, or “bob syrup”, of the soft confectionery is relatively viscous and has a higher density than the frappe component, and frequently contains a substantial amount of carbohydrate bulking agent such as a sugar/corn syrup blend or a polyol/hydrogenated starch hydrolysate blend. Conventionally, the final nougat composition is prepared by the addition of the “bob syrup” to the frappe under agitation, to form the basic nougat mixture. Further ingredients such as flavoring, additional carbohydrate bulking agent, colorants, preservatives, medicaments, mixtures thereof and the like may be added thereafter also under agitation. A general discussion of the composition and preparation of nougat confections may be found in B. W. Minifie, Chocolate, Cocoa and Confectionery: Science and Technology, 2nd edition, AVI Publishing Co., Inc., Westport, Conn. (1980), at pages 424-425, which disclosure is incorporated herein by reference.

The procedure for preparing the soft confectionery involves known procedures. In general, the frappe component is prepared first and thereafter the syrup component is slowly added under agitation at a temperature of at least about 65° C., and preferably at least about 100° C. The mixture of components is continued to be mixed to form a uniform mixture, after which the mixture is cooled to a temperature below 80° C., at which point, the flavor may be added. The mixture is further mixed for an additional period until it is ready to be removed and formed into suitable confectionery shapes.

Compressed tablet confectionery compositions contain particular materials and are formed into structures under pressure. These confections generally contain sugar, polyols, and optionally sugar substitutes, in amounts up to about 95%, by weight of the composition, and typical tablet excipients such as binders and lubricants.

The chewable confectionery product may include at least two separate and distinct segments: a dissolvable tablet core and a coating surrounding the core. The core may contain a first flavor, while the coating contains a second flavor. The two flavors may be the same, complementary or vastly different. Additional embodiments may have a third flavor in the coating. The three flavors may be the same, complementary or vastly different. The coating may be continuous as with, for example, a panned coating, or it may be particulate as with, for example, a sanded coating. The coating may be partial, i.e. only surrounding a portion of the core, or it may completely cover the core.

Other embodiments of chewable confectionery products may include a chewable candy core and a coating surrounding the core. The core may contain a first flavor, while the coating contains a second flavor. The two flavors may be the same, complementary or vastly different. Additional embodiments may have a third flavor in the coating. The three flavors may be the same, complementary or vastly different. The coating may be continuous as with, for example, a panned coating, or it may be particulate as with, for example, a sanded coating. The coating may be partial, i.e. only surrounding a portion of the core, or it may completely cover the core.

In accordance with some embodiments, the core of the confectionery product may be a pressed tablet. Alternatively, in some embodiments, the core may be any other form of tablet, such as an extruded tablet, molded tablet or any combination thereof. In some embodiments, the core may be water-soluble, particularly a water-soluble pressed tablet. The moisture content of the core may vary, but core generally may have a moisture content of less than about 1% or less. In addition, the core may have a hardness of at least about 10 KPU. In some embodiments, the core may be about 5% to about 95% by weight of the total confectionery product. In other embodiments, the core may be about 15% to 75% by weight. In addition to the first flavor, the core may include at least one bulk sweetener, such as a sugar sweetener and/or sugarless bulk sweetener. In pressed tablet embodiments, the bulk sweetener may be in a particulate form, such as a powder.

In some embodiments, the core of the confectionery product may be a chewy candy. Suitable chewy candies may include, but are not limited to, nougats, toffees, taffies, chewy candies, gummies, and jellies. In addition to the first flavor, the core may include at least one bulk sweetener, such as a sugar sweetener and/or sugarless bulk sweetener.

In some embodiments, the confectionery product may be a multi-region confection and may include a center-fill or first region, which may be a liquid or powder or other solid, or semi-solid, or gas, and a confectionery shell region. Some embodiments also may include an outer coating or third region, which typically provides a crunchiness to the piece when initially chewed. The outer coating or shell may at least partially surround the confectionery shell region. The outer coating or shell may be amorphous or crystalline and it may be continuous or particulate.

In some embodiments, the center-fill may be surrounded by a gummy candy shell. The center-fill of this embodiment may include isothiocyanates in the amount of about 0.005% to about 10% by weight of the center-fill region.

In some embodiments, the center-fill may be surrounded by a chewing gum shell. The center-fill of this embodiment may include isothiocyanates in the amount of about 0.05% to about 10% by weight of the center-fill region.

In some embodiments, the coating may be a particulate coating that is embedded into the surface of the confection while in other embodiments, the coating may be a particulate coating that adheres to the confection without being embedded. This process of surface treatment is also known as sanding, as described in U.S. application Ser. Nos. 11/738,779 and 11/738,845, each of which is incorporated herein by reference in its entirety. For example, in some embodiments, the coating composition may include an acid blend including lactic acid, tartaric acid, and fumaric acid. A sanding or coating composition may include the acid blend and a compound selected from 4-pentenyl isothiocyanate, 5-hexenyl isothiocyanate, 3-butenyl isothiocyanate, 3-methylthiopropyl isothiocyanate, and combinations thereof. The isothiocyanates may be present in amounts of about 0.001% to about 1.0% by weight of the sanding or coating composition. The coating compositions promote the perception of nasal clearing in the user.

In some embodiments, the confectionery shell region of the multi-region confection may include a hard candy shell. The hard candy shell may include 30% to about 80% glucose syrup and from about 15% to about 70% sucrose. In some embodiments, the hard candy shell may be sugarless and include any sugar-free sweetener known in the art. The hard candy shell may include flavors, sensates, colors, potentiators, and the like and combinations thereof.

In some embodiments, the confectionery shell region of the multi-region confection may include a gummy candy shell. The gummy candy shell composition may include any conventional gummy candy material such as, but not limited to, sweeteners, hydrocolloids, and food acids. Suitable examples of these materials are described above and may be used with equal applicability here. For the hydrocolloid materials, in some embodiments, a desired texture is created by using hydrocolloids that form chewable gels when combined with the other ingredients in the gummy candy shell composition.

In some embodiments, pectin and gelatin may be used together in a gummy candy shell composition as described in U.S. application Ser. No. 10/977,585, filed Oct. 28, 2004 and incorporated herein for all purposes. This combination of hydrocolloids can create a gummy candy shell that sets up quickly and provides adequate shell strength and desirable texture.

In some embodiments, the gummy candy shell composition may contain sweeteners in amounts from about 35% w/w to about 75% w/w of the gummy candy shell composition. In some embodiments, the gummy candy shell composition may contain from about 0.01% w/w to about 15% w/w, and preferably from about 1% to about 8% w/w of hydrocolloids. In some embodiments, the gummy candy shell composition may contain from about 0.3% to about 3%, and preferably from about 0.5% w/w and about 2.0% w/w food acids.

As with the liquid center-fill composition, in some embodiments, the gummy candy shell composition may also include buffering agents, coloring, flavoring, and preservatives.

Further, in some embodiments, the gummy candy shell composition may include functional ingredients such as, but not limited to, sensates, medicaments, nutrients such as vitamins and minerals and the like, nutraceuticals such as phytochemicals and the like, breath freshening agents, oral care agents, probiotic materials, prebiotic materials, taste and/or flavor potentiators, and throat care agents.

The gummy candy shell composition may be included in the coated liquid filled gummy candy composition in amounts from about 70% by weight of the total composition to about 90% by weight of the total composition.

The confectionery product may include a coating or shell and may at least substantially surround, or enrobe, the core. In some embodiments, the coating may wholly or completely surround the core. The coating may be about 5% to about 95% by weight of the total confectionery product. In other embodiments the core may be about 25% to about 85% by weight of the total confectionery product. The coating compositions may include a compound selected from 4-pentenyl isothiocyanate, 5-hexenyl isothiocyanate, 3-butenyl isothiocyanate, 3-methylthiopropyl isothiocyanate, and combinations thereof. The isothiocyanates may be present in amounts of about 0.001% to about 3.0% by weight of the coating composition. The coating compositions promote the perception of nasal clearing in the user.

In addition to the second flavor, the coating of the confectionery product also may contain at least one bulk sweetener. The bulk sweetener may be a sugar sweetener and/or sugarless bulk sweetener. In some embodiments, the coating may also contain a high intensity sweetener.

In addition to the unique attributes due to different flavor release profiles in the coating and the core, the crunchiness of the coating juxtaposed with the smoother chew of the core, can provide a unique sensory experience.

In some embodiments, an individual chewable confectionery product may weigh about 2 g to about 6 grams (g).

In general, the confectionery carrier is present in amounts of about 5% to about 99% by weight of the confectionery composition. More specifically, the confectionery carrier may be present in amounts of about 80% to about 99% by weight of the confectionery composition.

The confectionery compositions also may include amounts of conventional additives as set forth in the “Additional Components” section below.

### Chewing Gum Compositions

Some embodiments described herein provide confectionery compositions that include chewing gum compositions. These chewing gum confectionery compositions may include a confectionery carrier including an elastomer or chewing gum base and a compound selected from substituted or unsubstituted C₄₋₁₀ alkenyl isothiocyanates, 3-methylthiopropyl isothiocyanate, and combinations thereof. In other embodiments, the chewing gum confectionery composition may include a confectionery carrier including an elastomer or chewing gum base and a compound selected from 4-pentenyl isothiocyanate, 5-hexenyl isothiocyanate, 3-butenyl isothiocyanate, 3-methylthiopropyl isothiocyanate, and combinations thereof. The isothiocyanates may be present in amounts of about 0.001% to about 3.0% by weight of the chewing gum confectionery composition. The chewing gum confectionery compositions may optionally include sweetener(s) such as sugar and/or sugarless sweeteners. The chewing gum confectionery compositions promote the perception of nasal clearing in the user.

Chewing gum compositions may be provided in a variety of different forms, such as, for example, slab, pellet, sticks, balls, cubes, center-fill chewing gums, candy chewing gums, multi-layer chewing gums, deposited chewing gums and compressed chewing gums. The chewing gum compositions also may include at least one flavor and a variety of optional additives.

In some embodiments, the chewing gum product may be a multi-region chewing gum and may include a center-fill or first region, which may be a liquid or powder or other solid, or semi-solid, or gas, and a chewing gum shell region. Some embodiments also may include an outer coating or third region, which typically provides a crunchiness to the piece when initially chewed. The outer coating or shell may at least partially surround the chewing gum shell region. The outer coating or shell may be amorphous or crystalline and it may be continuous or particulate.

The chewing gum composition also may include a chewing gum base. The chewing gum base may include any component known in the chewing gum art. Such components may be water soluble, water-insoluble or a combination thereof. For example, the chewing gum base may include elastomers, bulking agents, waxes, elastomer solvents, emulsifiers, plasticizers, fillers and mixtures thereof.

The elastomers (rubbers) employed in the chewing gum base will vary greatly depending upon various factors such as the type of chewing gum base desired, the consistency of chewing gum composition desired and the other components used in the composition to make the final chewing gum product. The elastomer may be any water-insoluble polymer known in the art, and includes those chewing gum polymers utilized for chewing gums and bubble gums. Illustrative examples of suitable polymers in chewing gum bases include both natural and synthetic elastomers. For example, those polymers which are suitable in chewing gum base compositions include, without limitation, natural substances (of vegetable origin) such as chicle, natural rubber, crown gum, nispero, rosidinha, jelutong, perillo, niger gutta, tunu, balata, guttapercha, lechi capsi, sorva, gutta kay, and the like, and mixtures thereof. Examples of synthetic elastomers include, without limitation, styrene-butadiene copolymers (SBR), polyisobutylene, isobutylene-isoprene copolymers, polyethylene, polyvinyl acetate and the like, and mixtures thereof.

The amount of elastomer employed in the chewing gum base may vary depending upon various factors such as the type of chewing gum base used, the consistency of the chewing gum composition desired and the other components used in the composition to make the final chewing gum product. In general, the elastomer will be present in the chewing gum base in an amount from about 10% to about 60% by weight, desirably from about 35% to about 40% by weight.

In some embodiments, the chewing gum base may include wax. It softens the polymeric elastomer mixture and improves the elasticity of the chewing gum base. When present, the waxes employed will have a melting point below about 60° C., and preferably between about 45° C. and about 55° C. The low melting wax may be a paraffin wax. The wax may be present in the chewing gum base in an amount from about 6% to about 10%, and preferably from about 7% to about 9.5%, by weight of the chewing gum base.

In addition to the low melting point waxes, waxes having a higher melting point may be used in the chewing gum base in amounts up to about 5%, by weight of the chewing gum base. Such high melting waxes include beeswax, vegetable wax, candelilla wax, carnuba wax, most petroleum waxes, and the like, and mixtures thereof.

In addition to the components set out above, the chewing gum base may include a variety of other ingredients, such as components selected from elastomer solvents, emulsifiers, plasticizers, fillers, and mixtures thereof.

The chewing gum base may contain elastomer solvents to aid in softening the elastomer component. Such elastomer solvents may include those elastomer solvents known in the art, for example, terpinene resins such as polymers of alpha-pinene or beta-pinene, methyl, glycerol and pentaerythritol esters of rosins and modified rosins and gums such as hydrogenated, dimerized and polymerized rosins, and mixtures thereof. Examples of elastomer solvents suitable for use herein may include the pentaerythritol ester of partially hydrogenated wood and gum rosin, the pentaerythritol ester of wood and gum rosin, the glycerol ester of wood rosin, the glycerol ester of partially dimerized wood and gum rosin, the glycerol ester of polymerized wood and gum rosin, the glycerol ester of tall oil rosin, the glycerol ester of wood and gum rosin and the partially hydrogenated wood and gum rosin and the partially hydrogenated methyl ester of wood and rosin, and the like, and mixtures thereof. The elastomer solvent may be employed in the chewing gum base in amounts from about 2% to about 15%, and preferably from about 7% to about 11%, by weight of the chewing gum base.

The chewing gum base may also include emulsifiers which aid in dispersing the immiscible components into a single stable system. The emulsifiers useful in this invention include glyceryl monostearate, lecithin, fatty acid monoglycerides, diglycerides, propylene glycol monostearate, and the like, and mixtures thereof. The emulsifier may be employed in amounts from about 2% to about 15%, and more specifically, from about 7% to about 11%, by weight of the chewing gum base.

The chewing gum base may also include plasticizers or softeners to provide a variety of desirable textures and consistency properties. Because of the low molecular weight of these ingredients, the plasticizers and softeners are able to penetrate the fundamental structure of the chewing gum base making it plastic and less viscous. Useful plasticizers and softeners include lanolin, palmitic acid, oleic acid, stearic acid, sodium stearate, potassium stearate, glyceryl triacetate, glyceryl lecithin, glyceryl monostearate, propylene glycol monostearate, acetylated monoglyceride, glycerine, and the like, and mixtures thereof. Waxes, for example, natural and synthetic waxes, hydrogenated vegetable oils, petroleum waxes such as polyurethane waxes, polyethylene waxes, paraffin waxes, microcrystalline waxes, fatty waxes, sorbitan monostearate, tallow, propylene glycol, mixtures thereof, and the like, may also be incorporated into the chewing gum base. The plasticizers and softeners are generally employed in the chewing gum base in amounts up to about 20% by weight of the chewing gum base, and more specifically in amounts from about 9% to about 17%, by weight of the chewing gum base.

Plasticizers also include hydrogenated vegetable oils, such as soybean oil and cottonseed oils, which may be employed alone or in combination. These plasticizers provide the chewing gum base with good texture and soft chew characteristics. These plasticizers and softeners are generally employed in amounts from about 5% to about 14%, and more specifically in amounts from about 5% to about 13.5%, by weight of the chewing gum base.

Glycerin may also be employed as a softening agent, such as the commercially available United States Pharmacopeia (USP) grade. Glycerin is a syrupy liquid with a sweet warm taste and has a sweetness of about 60% of that of cane sugar. Because glycerin is hygroscopic, the glycerin may be maintained under anhydrous conditions throughout the preparation of the chewing gum composition.

In some embodiments, the chewing gum base may also include effective amounts of bulking agents such as mineral adjuvants which may serve as fillers and textural agents. Useful mineral adjuvants include calcium carbonate, magnesium carbonate, alumina, aluminum hydroxide, aluminum silicate, talc, tricalcium phosphate, dicalcium phosphate, calcium sulfate and the like, and mixtures thereof. These fillers or adjuvants may be used in the chewing gum base compositions in various amounts. Preferably the amount of filler, when used, will be present in an amount from about 15% to about 40%, and desirably from about 20% to about 30%, by weight of the chewing gum base.

A variety of traditional ingredients may be optionally included in the chewing gum base in effective amounts such as flavor agents and coloring agents, antioxidants, preservatives, and the like, some of which are described in more detail below in the section entitled “Additional Components”. For example, titanium dioxide and other dyes suitable for food, drug and cosmetic applications, known as F. D. & C. dyes, may be utilized. An anti-oxidant such as butylated hydroxytoluene (BHT), butylated hydroxyanisole (BHA), propyl gallate, vitamin E and mixtures thereof, may also be included. Other conventional chewing gum additives known to one having ordinary skill in the chewing gum art may also be used in the chewing gum base.

In general, the chewing gum base is present in amounts of about 5% to about 95% by weight of the chewing gum composition. More specifically, the chewing gum base may be present in amounts of about 20% to about 60% by weight of the chewing gum composition.

Chewing gum products may be prepared using standard techniques and equipment known to those skilled in the art. The apparatus useful in accordance with the embodiments described herein includes mixing and heating apparatus well known in the chewing gum manufacturing arts, and therefore the selection of the specific apparatus will be apparent to the artisan. For general chewing gum preparation processes see U.S. Pat. Nos. 4,271,197 to Hopkins et al., 4,352,822 to Cherukuri et al. and 4,497,832 to Cherukuri et al., each of which is incorporated herein by reference in its entirety.

In compressed chewing gum formats, the chewing gum base may be in a particulate form, such as, but not limited to, a powdered or granular chewing gum base, as opposed to molten or thermoplastic chewing gum base. The particulate chewing gum base may be essentially free of water and can readily be formed into any desired shape, such as by compression.

The particulate chewing gum base may be formed using standard grinding techniques known in the art. The starting material may be any conventional chewing gum base, such as those used to produce molten chewing gum bases. The particulate chewing gum base may be formed, for example, by shredding, grinding or crushing the chewing gum base or other processes, as described in U.S. Pat. Nos. 3,262,784, 4,405,647, 4,753,805 and 6,290,985 and U.S. Publication No. 2003/00276871, all of which are incorporated herein by reference in their entirety.

Desirably, the particulate chewing gum base is ground or the like into a particulate form that is similar in particle size to the tableting powder. By using components of like particle size, a homogenous mix of chewing gum base and tableting powder may be achieved, which may provide a chewing gum tablet of similar homogenous make-up. The chewing gum base and tableting powder may have a particle size of about 4 to about 100 mesh, desirably about 8 to about 25 mesh, and more desirably about 12 to about 20 mesh.

The particulate chewing gum base may be present in amounts of about 10% to about 80% by weight of the chewing gum composition, or tablet, desirably about 20% to about 50% by weight, and more desirably about 30% to about 40% by weight.

The particulate chewing gum base may be combined with a tableting powder to form the pressed chewing gum tablet. The tableting powder may be in a dry, finely-divided form. Desirable particle size is provided above. The tableting powder may be a sucrose-based, dextrose-based or polyol-based powder, or combinations thereof. For example, the polyol-based powder may be a sorbitol or mannitol powder. The tableting powder may include other optional ingredients, such as flavor agents, color agents, sugar and/or sugarless sweeteners, and the like and combinations thereof.

In some embodiments, it may be desirable to combine a food-grade lubricant with the particulate chewing gum base and tableting powder. Food-grade lubricants may assist in processing the chewing gum composition into pressed tablets. More specifically, lubricants are used to prevent excess wear on dies and punches in tableting manufacture. Lubricants may be useful immediately after compression of the tablet within the die to reduce friction between the tablet and inner die wall.

The food-grade lubricant may be added separately or it may be included with the tableting powder, as in some commercially available tableting powders. Examples of suitable food-grade lubricants include: metallic stearates; fatty acids; hydrogenated vegetable oil; partially hydrogenated vegetable oils; animal fats; polyethylene glycols; polyoxyethylene monostearate; talc; silicon dioxide; and combinations thereof. Food-grade lubricants may be present in amounts of about 0-6% by weight of the chewing gum composition.

Alternatively, in some embodiments, a compressible chewing gum composition may be formed by preparing a chewing gum composition and then grinding the mixture. The chewing gum composition may be prepared by mixing together molten chewing gum base, bulk sweeteners, softeners, plasticizers, other sweeteners, colors, and the like by any known mixing technique such as dough mixing. As with preparation of the particulate chewing gum base, the chewing gum mixture may be formed into a particulate chewing gum composition using standard grinding techniques known in the art. The particulate chewing gum may be formed, for example, by shredding, grinding or crushing the chewing gum or other processes, as described in U.S. Pat. Nos. 3,262,784, 4,405,647, 4,753,805 and 6,290,985 and U.S. Publication No. 2003/00276871, all of which are incorporated herein by reference in their entirety.

As described above, the compressible chewing gum composition may be in the form of a pressed chewing gum tablet. In some embodiments, the particulate chewing gum base and modified release ingredients are pressed into a tablet form. Upon chewing, the pressed chewing gum tablet consolidates into a soft chewy substance.

In some embodiments, the compressible chewing gum composition is a single-layer pressed tablet. In some embodiments, the compressible chewing gum composition is a multi-layer pressed tablet. Multi-layer tablet embodiments may have any desirable number of layers. Different layers may have the same or different thicknesses. In addition, different layers may include the same or different ingredients.

The pressed chewing gum tablet also may have a coating layer surrounding the tablet. The coating layer may contain any ingredients conventionally used in the chewing gum art. For instance, the coating may contain sugar, polyols or high intensity sweeteners or the like, coloring agents, active agents, flavor agents and warming and/or cooling agents, among others.

The compressible chewing gum compositions, or pressed tablets, desirably have a very low moisture content. In some embodiments, the tablets are essentially free of water. Accordingly, some embodiments have a total water content of greater than about 0% to about 5% by weight of the composition. The density of the composition, or tablet, may be about 0.2 to about 0.8 g/cc. Further, the compressible chewing gum compositions, or tablets, may have a dissolution rate of about 1 to about 20 minutes. When in a pressed tablet form, the chewing gum may have a Shore hardness of about 30 to about 200.

In contrast to dough mixed chewing gums where the chewing gum mixture can achieve temperatures of 35° C. to 60° C., compressed chewing gum temperatures can remain around ambient temperature (23° C. to 25° C.). In some embodiments, subjecting the compressible chewing gum compositions to lower temperatures can protect temperature sensitive ingredients from thermal degradation. Similarly, the absence of intimate mixing at temperatures above ambient can protect delivery systems that include temperature sensitive ingredients or ingredients subject to degradation from chewing gum ingredients such as flavors, plasticizers, and the like. Thus, ingredients susceptible to thermal or chemical degradation due to conventional dough mixing can be less likely to experience degradation in compressed chewing gum systems.

Any of the optional additives described below also may be included in the chewing gum compositions, as well as any conventional chewing gum additives known to those skilled in the art.

The chewing gum compositions may include amounts of conventional additives selected from the group consisting of sweetening agents, plasticizers, softeners, emulsifiers, waxes, fillers, bulking agents (carriers, extenders, bulk sweeteners), mineral adjuvants, flavor agents and coloring agents, antioxidants, acidulants, thickeners, medicaments, oral care actives, such as remineralization agents, antimicrobials and tooth whitening agents, as described in assignee's co-pending U.S. patent application Ser. No. 10/901,511, filed on Jul. 29, 2004 and entitled “Tooth Whitening Compositions and Delivery Systems Therefor,” which is incorporated herein by reference in its entirety, and the like, and mixtures thereof. Some of these additives may serve more than one purpose. For example, in sugarless chewing gum compositions, a sweetener, such as maltitol, isomalt, erythritol, xylitol, or other sugar alcohol, may also function as a bulking agent.

Bulk sweeteners include sugars, sugarless bulk sweeteners, or the like, or mixtures thereof. Bulk sweeteners generally are present in amounts of about 5% to about 99% by weight of the chewing gum composition. Suitable sugar sweeteners, sugarless bulk sweeteners and intense sweeteners may include any of those described in the section below entitled “Additional Components”.

The chewing gum composition may also include flavor agents, which may include any of those described below. The flavor agents generally may be present in chewing gum compositions in amounts from about 0.02% to about 5%, and more specifically from about 0.1% to about 4%, and even more specifically, from about 0.8% to about 3%, by weight of the composition.

The plasticizers, softening agents, mineral adjuvants, waxes and antioxidants discussed above, as being suitable for use in the chewing gum base, may also be used in the chewing gum composition. Examples of other conventional additives which may be used include emulsifiers, such as lecithin and glyceryl monostearate, thickeners, used alone or in combination with other softeners, such as methyl cellulose, alginates, carrageenan, xanthan gum, gelatin, carob, tragacanth, locust bean, and carboxy methyl cellulose, acidulants such as malic acid, adipic acid, citric acid, tartaric acid, fumaric acid, and mixtures thereof, and fillers, such as those discussed above under the category of mineral adjuvants.

Other conventional chewing gum additives known to one having ordinary skill in the chewing gum art also may be used in the chewing gum compositions.

Some embodiments extend to methods of preparing a chewing gum product. The products may be prepared using standard techniques and equipment known to those skilled in the art, which processes generally involve melting the chewing gum base, incorporating the desired ingredients while mixing and forming the batch into individual chewing gum pieces. The apparatus useful in accordance with the embodiments described herein includes mixing and heating apparatus well known in the chewing gum manufacturing arts, and therefore the selection of the specific apparatus will be apparent to the artisan. For general chewing gum preparation processes see U.S. Pat. Nos. 4,271,197 to Hopkins et al., 4,352,822 to Cherukuri et al. and 4,497,832 to Cherukuri et al., each of which is incorporated herein by reference in its entirety.

Individual chewing gum pieces may be formed using standard techniques known in the chewing gum art. For instance, chewing gum pieces may be prepared in the form of a slab, pellet, stick, balls, cubes, center-fill chewing gum, deposited chewing gum, compressed chewing gum or any other suitable format.

For instance, in some embodiments, a multi-region chewing gum may include a center-fill region, which may be a liquid or powder or other solid, or semi-solid, or gas, and a chewing gum region. Some embodiments also may include an outer chewing gum coating, which typically provides a crunchiness to the piece when initially chewed. The outer coating or shell may at least partially surround the chewing gum region. The outer coating or shell may be amorphous or crystalline and it may be continuous or particulate. Center-fill chewing gums and methods of preparing same are more fully described in assignee's co-pending U.S. patent application Ser. No. 10/925,822, filed on Aug. 24, 2004 and assignee's co-pending U.S. patent application Ser. No. 11/210,954, filed on Aug. 24, 2005, both entitled “Liquid-Filled Chewing Gum Composition,” the contents both of which are incorporated herein by reference.

The center-fill chewing gum composition and other compositions described herein may be formed by any technique known in the art which includes the method described by U.S. Pat. No. 6,280,780 to Degady et al. (“Degady”) which is herein incorporated by reference in its entirety. Degady describes an apparatus and method for forming center-filled chewing gum pellets. The method includes first extruding a liquid-filled rope of a chewing gum layer and passing the rope through a sizing mechanism including a series of pairs of pulley-shaped roller members. The roller members “size” the rope or strand of chewing gum material such that it leaves the series of rollers with the desired size and shape for entering a tablet-forming mechanism.

The rope is then led into a tablet-forming mechanism including a pair of rotating chain die members which are endless chain mechanisms and both rotate at the same speed by a motor and gear mechanism. Each of the chain mechanisms include a plurality of open curved die groove members which mate and form die cavities in which the pieces of chewing gum material (pellets or tablets) are formed. While Degady is limited to the formation of pellet or tablet shaped pieces, the chewing gum pieces may be of other shapes as described above. The shape of the die groove members may be altered to provided any desired shape.

The chewing gum may optionally be passed through a cooling tunnel either before entering the tablet-forming mechanism, after exiting the tablet-forming mechanism or both. Cooling of the rope prior to entering the tablet-forming mechanism may be beneficial to prevent rebound of the individual pieces and thus may provide an increase in productivity.

The cooled pieces of chewing gum material are then fed into a storage container for conditioning and further processing. At this point, the cooled pieces of chewing gum material could also be fed directly into a coating tunnel mechanism, such as a rotating tunnel mechanism.

Whether the pieces of formed chewing gum material are first stored, transported in a storage container, or fed directly into a coating tunnel or mechanism, the individual pieces of chewing gum material may subsequently be subjected to a conventional sugar or sugarless coating process in order to form a hard exterior shell on the liquid-filled chewing gum material. A variety of coating processes or mechanisms of this type are known. In some embodiments, the coating is applied in numerous thin layers of material in order to form an appropriate uniform coated and finished quality surface on the chewing gum products. The hard coating material, which may include sugar, maltitol, sorbitol or any other polyol, including those described herein, and optionally flavoring, is sprayed onto the pellets of chewing gum material as they pass through a coating mechanism or a coating tunnel and are tumbled and rotated therein. In addition, conditioned air is circulated or forced into the coating tunnel or mechanism in order to dry each of the successive coating layers on the formed products. In some embodiments, the coating, or outermost region, can be formed by lamination, dual or multiple extrusion, or any other process that creates an outermost region.

The coating composition may range from about 2% to about 80%, more specifically, about 20% to about 40% by weight of an individual chewing gum or confectionery piece which includes a center-fill, a chewing gum or confectionery region and a coating; even more specifically, from 25% to 35% and still more specifically around 30%. The coating may include sugar or polyol such as maltitol as the primary component, but may also include flavors, colors, and the like. as described below in the discussion of the chewing gum region. The coating or outermost region may be crystalline or amorphous.

Some other chewing gum embodiments may be in a compressed chewing gum format, such as, for example, a pressed tablet chewing gum. Such embodiments may include a particulate chewing gum base, which may include a compressible chewing gum base composition and a tableting powder. Compressed chewing gums are more fully described in assignee's co-pending PCT Application No. PCT/US2006/019761 and U.S. Provisional Application No. 60/734,680, filed on Nov. 8, 2005, and entitled “Compressible Gum System,” the contents of which are incorporated herein by reference.

In some embodiments, the chewing gum may have a coating thereon. Such coated chewing gums are typically referred to as pellet chewing gums. The outer coating may be hard or crunchy. The outer coating may be amorphous or crystalline. The coating may be continuous as with, for example, a panned coating, or it may be particulate as with, for example, a dusted or sanded coating. Any suitable coating material known to those skilled in the art may be employed. Typically, the outer coating may include sorbitol, maltitol, xylitol, isomalt, erythritol and other crystallizable polyols; sucrose may also be used. Furthermore the coating may include several opaque layers, such that the chewing gum composition is not visible through the coating itself, which can optionally be covered with a further one or more transparent layers for aesthetic, textural and protective purposes. The outer coating may also contain small amounts of water and gum arabic. The coating can be further coated with wax. The coating may be applied in a conventional manner by successive applications of a coating solution, with drying in between each coat. As the coating dries it usually becomes opaque and is usually white, though other colorants may be added. A polyol coating can be further coated with wax. The coating can further include colored flakes or speckles. If the composition includes a coating, it is possible that one or more oral care actives can be dispersed throughout the coating. This is especially preferred if one or more oral care actives is incompatible in a single phase composition with another of the actives. Flavors may also be added to yield unique product characteristics.

Other materials may be added to the coating to achieve desired properties. These materials may include without limitations, cellulosics such as carboxymethyl cellulose, gelatin, xanthan gum and gum arabic.

The coating composition may be applied by any method known in the art including the method described above. The coating composition may be present in an amount from about 2% to about 60%, more specifically from about 25% to about 45% by weight of the total chewing gum piece.

### Additional Components

The chewing gum and confectionery compositions may include a variety of optional additives, such as, but not limited to, sensates, mouth moistening agents, potentiators, sweeteners, effervescent agents, acids and flavors. A description of suitable additives follows.

### Sweetening Ingredients

Sweeteners can include sugars, sugarless bulk sweeteners, or the like, high intensity sweeteners, or mixtures thereof. Bulk sweeteners generally are present in amounts of about 5% to about 99% by weight of the confectionery or chewing gum base composition. Suitable sugar sweeteners generally include mono-saccharides, di-saccharides and poly-saccharides such as but not limited to, sucrose (sugar), dextrose, maltose, dextrin, xylose, ribose, glucose, mannose, galactose, fructose (levulose), invert sugar, corn syrups, maltodextrins, oligo saccharide syrups, fructo oligo saccharide syrups, partially hydrolyzed starch, corn syrup solids, resistant starches, and mixtures thereof.

Suitable sugarless bulk sweeteners include sugar alcohols (or polyols) such as, but not limited to, sorbitol, xylitol, mannitol, galactitol, maltitol, hydrogenated isomaltulose (ISOMALT), lactitol, erythritol, hydrogenated starch hydrolysate, and mixtures thereof

Suitable hydrogenated starch hydrolysates include those disclosed in U.S. Pat. No. 4,279,931 and various hydrogenated glucose syrups and/or powders which contain sorbitol, hydrogenated disaccharides, hydrogenated higher polysaccharides, or mixtures thereof. Hydrogenated starch hydrolysates are primarily prepared by the controlled catalytic hydrogenation of corn syrups. The resulting hydrogenated starch hydrolysates are mixtures of monomeric, dimeric, and polymeric saccharides. The ratios of these different saccharides give different hydrogenated starch hydrolysates different properties. Mixtures of hydrogenated starch hydrolysates, such as LYCASIN®, a commercially available product manufactured by Roquette Freres of France, and HYSTAR®, a commercially available product manufactured by SPI Polyols, Inc. of New Castle, Del., are also useful.

In some embodiments, high-intensity sweeteners also may be included as sweetening agents in the compositions. Without being limited to particular sweeteners, representative categories and examples include:

(a) water-soluble sweetening agents such as dihydrochalcones, monellin, steviosides, lo han quo, glycyrrhizin, dihydroflavenol, and L-aminodicarboxylic acid aminoalkenoic acid ester amides, such as those disclosed in U.S. Pat. No. 4,619,834, which disclosure is incorporated herein by reference, and mixtures thereof;

(b) water-soluble artificial sweeteners such as soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and mixtures thereof;

(c) dipeptide based sweeteners, such as L-aspartic acid derived sweeteners, such as L-aspartyl L-phenylalanine methyl ester (Aspartame), N[N-(3,3-dimethylbutyl)-L-α-aspartyl]-L-phenylalanine 1-methyl ester (Neotame), and materials described in U.S. Pat. No. 3,492,131, L-alphaaspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, and mixtures thereof;

(d) water-soluble sweeteners derived from naturally occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose or Splenda™; examples of chlorodeoxysucrose and chlorodeoxygalactosucrose derivatives include but are not limited to: 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose; 1,6-dichloro-1,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galacto-pyranosyl-1,6-dichloro-1,6-dideoxy-beta-D-fructofuranoside, or 4,6,1′,6′-tetrachloro-4,6,1′,6′-tetradeoxygalacto-sucrose; and 4,6,1′,6′-tetradeoxy-sucrose, and mixtures thereof;

(e) protein based sweeteners such as thaumaoccous danielli (Thaumatin I and II) and talin; and the sweetener monatin (2-hydroxy-2-(indol-3-ylmethyl)-4-aminoglutaric acid) and its derivatives.

The intense sweetening agents may be used in many distinct physical forms well-known in the art to provide an initial burst of sweetness and/or a prolonged sensation of sweetness. Without being limited thereto, such physical forms include free forms, spray dried forms, powdered forms, beaded forms, encapsulated forms, and mixtures thereof. In one embodiment, the sweetener is a high intensity sweetener such as aspartame, sucralose, and acesulfame potassium (e.g., Ace-K or acesulfame-K).

In some embodiments, the sweetener may be a polyol. Polyols can include, but are not limited to glycerol, sorbitol, maltitol, maltitol syrup, mannitol, isomalt, erythritol, xylitol, hydrogenated starch hydrolysates, polyglycidol syrups, polyglycidol powders, lactitol, and combinations thereof.

The sweetener may be used in amounts necessary to impart the desired effect associated with use of the sweetener. In general, an effective amount of intense sweetener may be utilized to provide the level of sweetness desired, and this amount may vary with the sweetener selected. The intense sweetener may be present in amounts from about 0.001% to about 3%, by weight of the composition, depending upon the sweetener or combination of sweeteners used. The exact range of amounts for each type of sweetener may be selected by those skilled in the art.

### Flavorants

In some embodiments, flavorants may include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof. Nonlimiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, apricot, banana, melon, apricot, ume, cherry, raspberry, blackberry, tropical fruit, mango, mangosteen, pomegranate, papaya and so forth. Other potential flavors whose release profiles can be managed include a milk flavor, a butter flavor, a cheese flavor, a cream flavor, and a yoghurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a chamomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bay leaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with the cooling agents, described herein below.

In some embodiments, other flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference. These may include natural as well as synthetic flavors.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, .e., melonal (melon), 2,6-dimethyloctanal (green fruit), and 2-dodecenal (citrus, mandarin), cherry, grape, blueberry, blackberry, strawberry shortcake, and mixtures thereof.

In some embodiments, flavoring agents are used at levels that provide a perceptible sensory experience i.e. at or above their threshold levels. In other embodiments, flavoring agents are used at levels below their threshold levels such that they do not provide an independent perceptible sensory experience. At subthreshold levels, the flavoring agents may provide an ancillary benefit such as flavor enhancement or potentiation.

In some embodiments, a flavoring agent may be employed in either liquid form and/or dried form. When employed in the latter form, suitable drying means such as spray drying the liquid may be used. Alternatively, the flavoring agent may be absorbed onto water soluble materials, such as cellulose, starch, sugar, maltodextrin, gum arabic and so forth or may be encapsulated. In still other embodiments, the flavoring agent may be adsorbed onto silicas, zeolites, and the like.

In some embodiments, the flavoring agents may be used in many distinct physical forms. Without being limited thereto, such physical forms include free forms, such as spray dried, powdered, beaded forms, encapsulated forms, and mixtures thereof.

Illustrations of the encapsulation of flavors as well as other additional components can be found in the examples provided herein. Typically, encapsulation of a component will result in a delay in the release of the predominant amount of the component during consumption of a confectionery composition that includes the encapsulated component (e.g., as part of a delivery system added as an ingredient to the chewing confectionery composition). In some embodiments, the release profile of the ingredient (e.g., the flavor, sweetener, and the like and combinations thereof.) can be managed by managing various characteristics of the ingredient, delivery system containing the ingredient, and/or the confectionery composition containing the delivery system and/or how the delivery system is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the confectionery composition, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, and the like.

In general, the at least one flavor is present in amounts of about 0.1% to about 15% by weight of the confectionery composition. More specifically, flavors may be present in amounts of about 0.5% to about 5.0% by weight of the confectionery compositions.

### Coloring Ingredients

In some embodiments, one or more colors can be included. As classified by the United States Food, Drug, and Cosmetic Act (21 C.F.R. 73), colors can include exempt from certification colors (sometimes referred to as natural even though they can be synthetically manufactured) and certified colors (sometimes referred to as artificial), or combinations thereof. In some embodiments, exempt from certification or natural colors can include, but are not limited to annatto extract, (E160b), bixin, norbixin, astaxanthin, dehydrated beets (beet powder), beetroot red/betanin (E162), ultramarine blue, canthaxanthin (E161g), cryptoxanthin (E161c), rubixanthin (E161d), violanxanthin (E161e), rhodoxanthin (E161f), caramel (E150(a-d)), β-apo-8′-carotenal (E160e), β-carotene (E160a), alpha carotene, gamma carotene, ethyl ester of beta-apo-8 carotenal (E160f), flavoxanthin (E161a), lutein (E161b), cochineal extract (E120); carmine (E132), carmoisine/azorubine (E122), sodium copper chlorophyllin (E141), chlorophyll (E140), toasted partially defatted cooked cottonseed flour, ferrous gluconate, ferrous lactate, grape color extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot oil, corn endosperm oil, paprika, paprika oleoresin, phaffia yeast, riboflavin (E101), saffron, titanium dioxide, turmeric (E100), turmeric oleoresin, amaranth (E123), capsanthin/capsorbin (E160c), lycopene (E160d), and combinations thereof.

In some embodiments, certified colors can include, but are not limited to, FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (E110), ponceau (E124), erythrosine (E127), patent blue V (E131), titanium dioxide (E171), aluminium (E173), silver (E174), gold (E175), pigment rubine/lithol rubine BK (E180), calcium carbonate (E170), carbon black (E153), black PN/brilliant black BN (E151), green S/acid brilliant green BS (E142), and combinations thereof. In some embodiments, certified colors can include FD&C aluminum lakes. These consist of the aluminum salts of FD&C dyes extended on an insoluble substrate of alumina hydrate. Additionally, in some embodiments, certified colors can be included as calcium salts.

### Sensate Ingredients

Sensate compounds can include cooling agents, warming agents, tingling agents, effervescent agents, and combinations thereof. A variety of well known cooling agents may be employed. For example, among the useful cooling agents are included xylitol, erythritol, dextrose, sorbitol, menthol, menthane, menthone, ketals, menthone ketals, menthone glycerol ketals, substituted p-menthanes, acyclic carboxamides, mono menthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N,2,3-trimethyl-2-isopropyl butanamide (WS-23), N-ethyl-p-menthane-3-carboxamide (WS-3), ethyl ester of N—[[5-methyl-2-(1-methylethyl)cyclohexyl]carbonyl]glycine (WS5), as well as the substantially pure ethyl ester of N—[[5-methyl-2-(1-methylethyl)cyclohexyl]carbonyl]glycine as disclosed in U.S. Pat. No. 7,189,760 to Erman, et al. which is incorporated in its entirety herein by reference, isopulegol, menthyloxy propane diol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, N,2,3-trimethyl-2-(1-methylethyl)-butanamide, n-ethyl-t-2-c-6 nonadienamide, N,N-dimethyl menthyl succinamide, substituted p-menthanes, substituted p-menthane-carboxamides, 2-isopropanyl-5-methylcyclohexanol (from Hisamitsu Pharmaceuticals, hereinafter “isopregol”); menthone glycerol ketals (FEMA 3807, tradename FRESCOLAT® type MGA); 3-1-menthoxypropane-1,2-diol (from Takasago, FEMA 3784); and menthyl lactate; (from Haarman & Reimer, FEMA 3748, tradename FRESCOLAT® type ML), WS-30, WS-14, Eucalyptus extract (p-Mehtha-3,8-Diol), Menthol (its natural or synthetic derivatives), Menthol PG carbonate, Menthol EG carbonate, Menthol glyceryl ether, N-tertbutyl-p-menthane-3-carboxamide, P-menthane-3-carboxylic acid glycerol ester, Methyl-2-isopropyl-bicyclo (2.2.1), Heptane-2-carboxamide; Menthol methyl ether, menthyl pyrrolidone carboxylate, cyclic α-keto enamines, cyclotene derivatives such as cyclopentenes including 3-methyl-2-(1-pyrrolidinyl)-2-cyclopenten-1-one and 5-methyl-2-(1-pyrrolidinyl)-2-cyclopenten-1-one, compounds of the formula:

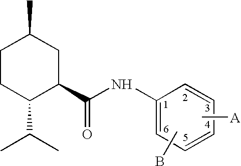

wherein B is selected from H, C3, C2H5, OCH3, 0C2H5; and OH; and wherein A is a moiety of the formula-CO-D, wherein D is selected from the following moieties: (i)—NR1R2, wherein R′ and R2 are independently selected from H and C1-C8 straight or branched-chain aliphatic, alkoxyalkyl, hydroxyalkyl, araliphatic and cycloalkyl groups, or R1 and R2 together with the nitrogen atom to which they are attached form part of an optionally-substituted, five- or six-membered heterocyclic ring; (ii)-NIICH2COOCH2CH3,—NHCH2CONH2,—NHCH2CH2OCH3,—NHCH2CH2OH,—NHCH2CH(OH)CH2OH and (iii) a moiety selected from the group consisting of:

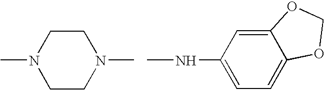

as disclosed in PCT Patent Application WO2006/125334 to Bell et al. which is incorporated in its entirety herein by reference, among others. These and other suitable cooling agents are further described in the following U.S. patents, all of which are incorporated in their entirety by reference hereto: U.S. Pat. Nos. 4,230,688; 4,032,661; 4,459,425; 4,178,459; 4,296,255; 4,136,163; 5,009,893; 5,266,592; 5,698,181; 6,277,385; 6,627,233; 7,030,273. Still other suitable cooling agents are further described in the following U.S. Patent Applications, all of which are incorporated in their entirety by reference hereto: U.S. 2005/0222256; 2005/0265930.

In some embodiments, warming components may be selected from a wide variety of compounds known to provide the sensory signal of warming to the user. These compounds offer the perceived sensation of warmth, particularly in the oral cavity, and often enhance the perception of flavors, sweeteners and other organoleptic components. In some embodiments, useful warming compounds can include vanillyl alcohol n-butylether (TK-1000) supplied by Takasago Perfumary Company Limited, Tokyo, Japan, vanillyl alcohol n-propylether, vanillyl alcohol isopropylether, vanillyl alcohol isobutylether, vanillyl alcohol n-aminoether, vanillyl alcohol isoamyleather, vanillyl alcohol n-hexyleather, vanillyl alcohol methylether, vanillyl alcohol ethylether, gingerol, shogaol, paradol, zingerone, capsaicin, dihydrocapsaicin, nordihydrocapsaicin, homocapsaicin, homodihydrocapsaicin, ethanol, isopropyl alcohol, iso-amylalcohol, benzyl alcohol, glycerine, and combinations thereof.

In some embodiments, a tingling sensation can be provided. One such tingling sensation is provided by adding jambu, oleoresin, or spilanthol to some examples. In some embodiments, alkylamides extracted from materials such as jambu or sanshool can be included. In some embodiments, a tingling sensation is provided by mouth moistening ingredients such as those mentioned below including, but not limited to, trans-pellitorin and alkadienamides. Additionally, in some embodiments, a sensation is created due to effervescence. Such effervescence is created by combining an alkaline material with an acidic material. In some embodiments, an alkaline material can include alkali metal carbonates, alkali metal bicarbonates, alkaline earth metal carbonates, alkaline earth metal bicarbonates and mixtures thereof. In some embodiments, an acidic material can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid, aspartic acid, benzoic acid, caffeotannic acid, iso-citric acid, citramalic acid, galacturonic acid, glucuronic acid, glyceric acid, glycolic acid, ketoglutaric acid, a-ketoglutaric acid, lactoisocitric acid, oxalacetic acid, pyruvic acid, quinic acid, shikimic acid, succinic acid, tannic acid, hydroxyacetic acid, suberic acid, sebacic acid, azelaic acid, pimelic acid, capric cid, and combinations thereof. Examples of “tingling” type sensates can be found in U.S. Pat. No. 6,780,443, the entire contents of which are incorporated herein by reference for all purposes.

Sensate components may also be referred to as “trigeminal stimulants” such as those disclosed in U.S. Patent Application No. 2005/0202118, which is incorporated herein by reference. Trigeminal stimulants are defined as an orally consumed product or agent that stimulates the trigeminal nerve. Examples of cooling agents which are trigeminal stimulants include menthol, WS-3, N-substituted p-menthane carboxamide, acyclic carboxamides including WS-23, WS-5, WS-14, methyl succinate, and menthone glycerol ketals. Trigeminal stimulants can also include flavors, tingling agents, Jambu extract, vanillyl alkyl ethers, such as vanillyl n-butyl ether, spilanthol, Echinacea extract, Northern Prickly Ash extract, capsaicin, capsicum oleoresin, red pepper oleoresin, black pepper oleoresin, piperine, ginger oleoresin, gingerol, shoagol, cinnamon oleoresin, cassia oleoresin, cinnamic aldehyde, eugenol, cyclic acetal of vanillin and menthol glycerin ether, unsaturated amides, and combinations thereof. Other cooling compounds can include derivatives of 2,3-dimethyl-2-isopropylbutyric acid such as those disclosed in U.S. Pat. No. 7,030,273, which is incorporated herein by reference.

In addition to trigeminal nerve stimulants and cooling compounds, a cooling sensation can be provided by materials exhibiting a negative heat of solution including, but not limited to, polyols such as xylitol, erythritol, dextrose, and sorbitol, and combinations thereof.

In some embodiments, sensate components are used at levels that provide a perceptible sensory experience, i.e. at or above their threshold levels. In other embodiments, sensate components are used at levels below their threshold levels such that they do not provide an independent perceptible sensory experience. At subthreshold levels, the sensates may provide an ancillary benefit such as flavor or sweetness enhancement or potentiation.

### Breath Freshening Ingredients

Breath fresheners can include essential oils as well as various aldehydes, alcohols, and similar materials. In some embodiments, essential oils can include oils of spearmint, peppermint, wintergreen, sassafras, chlorophyll, citral, geraniol, cardamom, clove, sage, carvacrol, eucalyptus, cardamom, magnolia bark extract, marjoram, cinnamon, lemon, lime, grapefruit, and orange. In some embodiments, aldehydes such as cinnamic aldehyde and salicylaldehyde can be used. Additionally, chemicals such as menthol, carvone, iso-garrigol, and anethole can function as breath fresheners. Of these, the most commonly employed are oils of peppermint, spearmint and chlorophyll.

In addition to essential oils and chemicals derived from them, in some embodiments breath fresheners can include but are not limited to zinc citrate, zinc acetate, zinc fluoride, zinc ammonium sulfate, zinc bromide, zinc iodide, zinc chloride, zinc nitrate, zinc fluorosilicate, zinc gluconate, zinc tartarate, zinc succinate, zinc formate, zinc chromate, zinc phenol sulfonate, zinc dithionate, zinc sulfate, silver nitrate, zinc salicylate, zinc glycerophosphate, copper nitrate, chlorophyll, copper chlorophyll, chlorophyllin, hydrogenated cottonseed oil, chlorine dioxide, beta cyclodextrin, zeolite, silica-based materials, carbon-based materials, enzymes such as laccase, and combinations thereof.

In some embodiments, the release profiles of probiotics can be managed for a confectionery including, but not limited to lactic acid producing microorganisms such as Bacillus coagulans, Bacillus subilis, Bacillus lalerosporus, Bacillus laevolacticus, Sporolactobacillus inulinus, Lactobacillus acidophilus, Lactobacillus curvatus, Lactobacillus plantarum, Lactobacillus jenseni, Lactobacillus casei, Lactobacillus fermentum, Lactococcus lactis, Pedioccocus acidilacti, Pedioccocus pentosaceus, Pedioccocus urinae, Leuconostoc mesenieroides, Bacillus coagulans, Bacillus subtilis, Bacillus laterosporus, Bacillus laevolacticus, Sporolactobacillus inulinus and mixtures thereof. Breath fresheners are also known by the following trade names: Retsyn,™ Actizol,™ and Nutrazin™. Examples of malodor-controlling compositions are also included in U.S. Pat. No. 5,300,305 to Stapler et al. and in U.S. Patent Application Publication Nos. 2003/0215417 and 2004/0081713 which are incorporated in their entirety herein by reference for all purposes.

### Dental Care Ingredients

Dental care ingredients (also known as oral care ingredients) may include but are not limited to tooth whiteners, stain removers, oral cleaning, bleaching agents, desensitizing agents, dental remineralization agents, antibacterial agents, anticaries agents, plaque acid buffering agents, surfactants and anticalculus agents. Non-limiting examples of such ingredients can include, hydrolytic agents including proteolytic enzymes, abrasives such as hydrated silica, calcium carbonate, sodium bicarbonate and alumina, other active stain-removing components such as surface-active agents, including, but not limited to anionic surfactants such as sodium stearate, sodium palminate, sulfated butyl oleate, sodium oleate, salts of fumaric acid, glycerol, hydroxylated lecithin, sodium lauryl sulfate and chelators such as polyphosphates, which are typically employed as tartar control ingredients. In some embodiments, dental care ingredients can also include tetrasodium pyrophosphate and sodium tri-polyphosphate, sodium bicarbonate, sodium acid pyrophosphate, sodium tripolyphosphate, xylitol, sodium hexametaphosphate.

In some embodiments, peroxides such as carbamide peroxide, calcium peroxide, magnesium peroxide, sodium peroxide, hydrogen peroxide, and peroxydiphospate are included. In some embodiments, potassium nitrate and potassium citrate are included. Other examples can include casein glycomacropeptide, calcium casein peptone-calcium phosphate, casein phosphopeptides, casein phosphopeptide-amorphous calcium phosphate (CPP-ACP), and amorphous calcium phosphate. Still other examples can include papaine, krillase, pepsin, trypsin, lysozyme, dextranase, mutanase, glycoamylase, amylase, glucose oxidase, and combinations thereof.

Further examples can include surfactants such as sodium stearate, sodium ricinoleate, and sodium lauryl sulfate surfactants for use in some embodiments to achieve increased prophylactic action and to render the dental care ingredients more cosmetically acceptable. Surfactants can preferably be detersive materials which impart to the composition detersive and foaming properties. Suitable examples of surfactants are water-soluble salts of higher fatty acid monoglyceride monosulfates, such as the sodium salt of the monosulfated monoglyceride of hydgrogenated coconut oil fatty acids, higher alkyl sulfates such as sodium lauryl sulfate, alkyl aryl sulfonates such as sodium dodecyl benzene sulfonate, higher alkyl sulfoacetates, sodium lauryl sulfoacetate, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine.

In addition to surfactants, dental care ingredients can include antibacterial agents such as, but not limited to, triclosan, chlorhexidine, zinc citrate, silver nitrate, copper, limonene, and cetyl pyridinium chloride. In some embodiments, additional anticaries agents can include fluoride ions or fluorine-providing components such as inorganic fluoride salts. In some embodiments, soluble alkali metal salts, for example, sodium fluoride, potassium fluoride, sodium fluorosilicate, ammonium fluorosilicate, sodium monofluorophosphate, as well as tin fluorides, such as stannous fluoride and stannous chloride can be included. In some embodiments, a fluorine-containing compound having a beneficial effect on the care and hygiene of the oral cavity, e.g., diminution of enamel solubility in acid and protection of the teeth against decay may also be included as an ingredient. Examples thereof include sodium fluoride, stannous fluoride, potassium fluoride, potassium stannous fluoride (SnF₂—KF), sodium hexafluorostannate, stannous chlorofluoride, sodium fluorozirconate, and sodium monofluorophosphate. In some embodiments, urea is included.

Further examples are included in the following U.S. patents and U.S. published patent applications, the contents of all of which are incorporated in their entirety herein by reference for all purposes: U.S. Pat. Nos. 5,227,154 to Reynolds, 5,378,131 to Greenberg, 6,846,500 to Luo et al., 6,733,818 to Luo et al., 6,696,044 to Luo et al., 6,685,916 to Holme et al., 6,485,739 to Luo et al., 6,479,071 to Holme et al., 6,471,945 to Luo et al., U.S. Patent Publication Nos. 2005/0025721 to Holme et al., 2005/0008732 to Gebreselassie et al., and 2004/0136928 to Holme et al.

### Active Ingredients

Actives generally refer to those ingredients that are included in a delivery system and/or confectionery composition for the desired end benefit they provide to the user. In some embodiments, actives can include medicaments, nutrients, nutraceuticals, herbals, nutritional supplements, pharmaceuticals, drugs, and the like and combinations thereof.

Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies such as sildenafil citrate, which is currently marketed as Viagra™, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids such as bromocryptine or nicotine, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of active ingredients contemplated for use in some embodiments can include antacids, H2-antagonists, and analgesics. For example, antacid dosages can be prepared using the ingredients calcium carbonate alone or in combination with magnesium hydroxide, and/or aluminum hydroxide. Moreover, antacids can be used in combination with H2-antagonists.

Analgesics include opiates and opiate derivatives, such as Oxycontin™, ibuprofen, aspirin, acetaminophen, and combinations thereof that may optionally include caffeine.

Other drug active ingredients for use in embodiments can include anti-diarrheals such as Immodium™ AD, anti-histamines, anti-tussives, decongestants, vitamins, and breath fresheners. Also contemplated for use herein are anxiolytics such as Xanax™; anti-psychotics such as Clozaril™ and Haldol™; non-steroidal anti-inflammatories (NSAID's) such as ibuprofen, naproxen sodium, Voltaren™ and Lodine™, anti-histamines such as Claritin™, Hismanal™, Relafen™, and Tavist™; anti-emetics such as Kytril™ and Cesamet™; bronchodilators such as Bentolin™, Proventil™; anti-depressants such as Prozac™, Zoloft™, and Paxil™; anti-migraines such as Imigra™, ACE-inhibitors such as Vasotec™, Capoten™ and Zestril™; anti-Alzheimer's agents, such as Nicergoline™; and CaH-antagonists such as Procardia™, Adalat™, and Calan™.

The popular H2-antagonists which are contemplated for use in the present invention include cimetidine, ranitidine hydrochloride, famotidine, nizatidien, ebrotidine, mifentidine, roxatidine, pisatidine and aceroxatidine.

Active antacid ingredients can include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

A variety of nutritional supplements may also be used as active ingredients including virtually any vitamin or mineral. For example, vitamin A, vitamin C, vitamin D, vitamin E, vitamin K, vitamin B₆, vitamin B₁₂, thiamine, riboflavin, biotin, folic acid, niacin, pantothenic acid, sodium, potassium, calcium, magnesium, phosphorus, sulfur, chlorine, iron, copper, iodine, zinc, selenium, manganese, choline, chromium, molybdenum, fluorine, cobalt and combinations thereof, may be used.

Examples of nutritional supplements that can be used as active ingredients are set forth in U.S. Patent Application Publication Nos. 2003/0157213 A1, 2003/0206993 and 2003/0099741 A1 which are incorporated in their entirety herein by reference for all purposes.

Various herbals may also be used as active ingredients such as those with various medicinal or dietary supplement properties. Herbals are generally aromatic plants or plant parts and or extracts thereof that can be used medicinally or for flavoring. Suitable herbals can be used singly or in various mixtures. Commonly used herbs include Echinacea, Goldenseal, Calendula, Rosemary, Thyme, Kava Kava, Aloe, Blood Root, Grapefruit Seed Extract, Black Cohosh, Ginseng, Guarana, Cranberry, Gingko Biloba, St. John's Wort, Evening Primrose Oil, Yohimbe Bark, Green Tea, Ma Huang, Maca, Bilberry, Lutein, and combinations thereof.

### Effervescing System Ingredients

An effervescent system may include one or more edible acids and one or more edible alkaline materials. The edible acid(s) and the edible alkaline material(s) may react together to generate effervescence.

In some embodiments, the alkaline material(s) may be selected from, but is not limited to, alkali metal carbonates, alkali metal bicarbonates, alkaline earth metal carbonates, alkaline earth metal bicarbonates, and combinations thereof. The edible acid(s) may be selected from, but is not limited to, citric acid, phosphoric acid, tartaric acid, malic acid, ascorbic acid, and combinations thereof. In some embodiments, an effervescing system may include one or more other ingredients such as, for example, carbon dioxide, oral care ingredients, flavorants, and the like and combinations thereof.

For examples of use of an effervescing system in a chewing confectionery, refer to U.S. Provisional Patent No. 60/618,222 filed Oct. 13, 2004, and entitled “Effervescent Pressed Confectionery Tablet Compositions,” the contents of which are incorporated herein by reference for all purposes. Other examples can be found in U.S. Pat. No. 6,235,318, the contents of which are incorporated herein by reference for all purposes.

### Appetite Suppressor Ingredients

Appetite suppressors can be ingredients such as fiber and protein that function to depress the desire to consume food. Appetite suppressors can also include benzphetamine, diethylpropion, mazindol, phendimetrazine, phenternine, hoodia (P57), Olibra™, ephedra, caffeine and combinations thereof. Appetite suppressors are also known by the following trade names: Adipex,™ Adipost,™ Bontril™ PDM, Bontril™ Slow Release, Didrex,™ Fastin,™ Ionamin,™ Mazanor,™ Melfiat,™ Obenix,™ Phendiet,™ Phendiet-105™ Phentercot,™ Phentride,™ Plegine,™ Prelu-2™ Pro-Fast™ PT 105,™ Sanorex,™ Tenuate,™ Sanorex,™ Tenuate,™ Tenuate Dospan,™ Tepanil Ten-Tab,™ Teramine,™ and Zantryl,™ . These and other suitable appetite suppressors are further described in the following U.S. patents, all of which are incorporated in their entirety by reference hereto: U.S. Pat. No. 6,838,431 to Portman, U.S. Pat. No. 6,716,815 to Portman, U.S. Pat. No. 6,558,690 to Portman, U.S. Pat. No. 6,468,962 to Portman, U.S. Pat. No. 6,436,899 to Portman.

### Potentiator Ingredients

Potentiators can consist of materials that may intensify, supplement, modify or enhance the taste and/or aroma perception of an original material without introducing a characteristic taste and/or aroma perception of their own. In some embodiments, potentiators designed to intensify, supplement, modify, or enhance the perception of flavor, sweetness, tartness, umami, kokumi, saltiness and combinations thereof can be included.

In some embodiments, examples of suitable potentiators, also known as taste potentiators include, but are not limited to, neohesperidin dihydrochalcone, optically active S-alkyl 2-methyl butane thioate compounds such as those disclosed in PCT Application Number WO 2007/032262 to Ogura et al., which is incorporated in its entirety herein by reference, chlorogenic acid, alapyridaine, cynarin, miraculin, glupyridaine, pyridinium-betain compounds, glutamates, such as monosodium glutamate and monopotassium glutamate, neotame, thaumatin, tagatose, trehalose, salts, such as sodium chloride, monoammonium glycyrrhizinate, vanilla extract (in ethyl alcohol), sugar acids, potassium chloride, sodium acid sulfate, hydrolyzed vegetable proteins, hydrolyzed animal proteins, yeast extracts, adenosine monophosphate (AMP), glutathione, nucleotides, such as inosine monophosphate, disodium inosinate, xanthosine monophosphate, guanylate monophosphate, alapyridaine (N-(1-carboxyethyl)-6-(hydroxymethyl)pyridinium-3-ol inner salt, compositions comprising 5′-nucleotides such as those disclosed in US 2006/0078972 to Noordam et al., which is incorporated in its entirety herein by reference, sugar beet extract (alcoholic extract), sugarcane leaf essence (alcoholic extract), curculin, strogin, mabinlin, gymnemic acid, hydroxybenzoic acids, 3-hydrobenzoic acid, 2,4-dihydrobenzoic acid, citrus aurantium, vanilla oleoresin, sugarcane leaf essence, maltol, ethyl maltol, vanillin, licorice glycyrrhizinates, compounds that respond to the TRPM5 ion channel that mediates taste receptors for sweet, bitter, and savory tastes as disclosed in U.S. Patent Application Number 2005/0019830 to Penner et al., which is incorporated in its entirety herein by reference, pyridinium betain compounds as disclosed in U.S. Pat. No. 7,175,872 to Hofmann et al., which is incorporated in its entirety herein by reference, compounds that respond to G-protein coupled receptors (T2Rs and T1Rs) and taste potentiator compositions that impart kokumi, as disclosed in U.S. Pat. No. 5,679,397 to Kuroda et al., which is incorporated in its entirety herein by reference. “Kokumi” refers to materials that impart “mouthfulness” and “good body”.

Sweetener potentiators, which are a type of taste potentiator, enhance the taste of sweetness. In some embodiments, exemplary sweetener potentiators include, but are not limited to, monoammonium glycyrrhizinate, licorice glycyrrhizinates, citrus aurantium, alapyridaine, alapyridaine (N-(1-carboxyethyl)-6-(hydroxymethyl)pyridinium-3-ol) inner salt, miraculin, curculin, strogin, mabinlin, gymnemic acid, cynarin, glupyridaine, pyridinium-betain compounds, sugar beet extract, neotame, thaumatin, neohesperidin dihydrochalcone, hydroxybenzoic acids, tagatose, trehalose, maltol, ethyl maltol, vanilla extract, vanilla oleoresin, vanillin, sugar beet extract (alcoholic extract), sugarcane leaf essence (alcoholic extract), compounds that respond to G-protein coupled receptors (T2Rs and T1Rs) and combinations thereof.

Additional examples of potentiators for the enhancement of salt taste include acidic peptides, such as those disclosed in U.S. Pat. No. 6,974,597, herein incorporated by reference. Acidic peptides include peptides having a larger number of acidic amino acids, such as aspartic acid and glutamic acid, than basic amino acids, such as lysine, arginine and histidine. The acidic peptides are obtained by peptide synthesis or by subjecting proteins to hydrolysis using endopeptidase, and if necessary, to deamidation. Suitable proteins for use in the production of the acidic peptides or the peptides obtained by subjecting a protein to hydrolysis and deamidation include plant proteins, (e.g. wheat gluten, corn protein (e.g., zein and gluten meal), soybean protein isolate), animal proteins (e.g., milk proteins such as milk casein and milk whey protein, muscle proteins such as meat protein and fish meat protein, egg white protein and collagen), and microbial proteins (e.g., microbial cell protein and polypeptides produced by microorganisms).

The sensation of warming or cooling effects may also be prolonged with the use of a hydrophobic sweetener as described in U.S. Patent Application Publication 2003/0072842 A1 which is incorporated in its entirety herein by reference. For example, such hydrophobic sweetners include those of the formulae I-XI as set forth below:

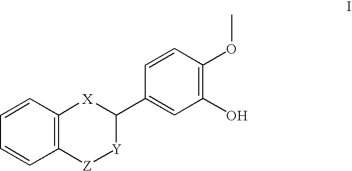

wherein X, Y and Z are selected from the group consisting of CH₂, O and S;

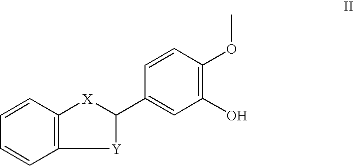

wherein X and Y are selected from the group consisting of S and O;

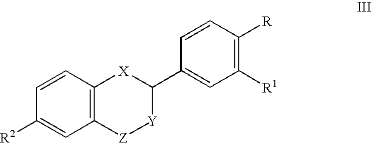

wherein X is S or O; Y is O or CH₂; Z is CH₂, SO₂ or S; R is OCH₃, OH or H; R¹ is SH or OH and R² is H or OH;

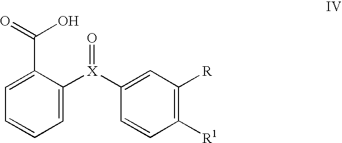

wherein X is C or S; R is OH or H and R¹ is OCH₃or OH;

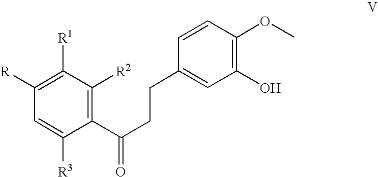

wherein R, R² and R³ are OH or H and R¹ is H or COOH;

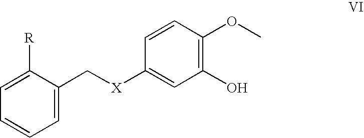

wherein X is O or CH₂ and R is COOH or H;

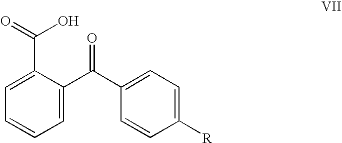

wherein R is CH₃CH₂, OH, N(CH3)₂ or Cl;

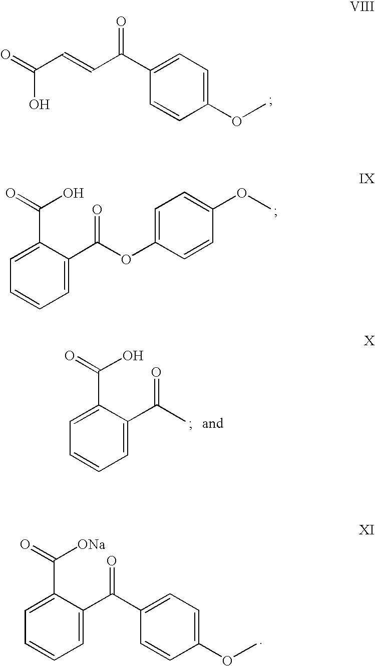

Perillartine may also be added as described in U.S. Pat. No. 6,159,509 also incorporated in its entirety herein by reference.

### Food Acid Ingredients

Acids can include, but are not limited to acetic acid, adipic acid, ascorbic acid, butyric acid, citric acid, fumaric acid, fumaric acid, glyconic acid, lactic acid, phosphoric acid, malic acid, oxalic acid, succinic acid, tartaric acid, aspartic acid, benzoic acid, caffeotannic acid, iso-citric acid, citramalic acid, galacturonic acid, glucuronic acid, glyceric acid, glycolic acid, ketoglutaric acid, a-ketoglutaric acid, lactoisocitric acid, oxalacetic acid, pyruvic acid, quinic acid, shikimic acid, succinic acid, tannic acid, hydroxyacetic acid, suberic acid, sebacic acid, azelaic acid, pimelic acid, capric cid, and combinations thereof.

### Micronutrient Ingredients

Micronutrients can include materials that have an impact on the nutritional well being of an organism even though the quantity required by the organism to have the desired effect is small relative to macronutrients such as protein, carbohydrate, and fat. Micronutrients can include, but are not limited to vitamins, minerals, enzymes, phytochemicals, antioxidants, and combinations thereof.

In some embodiments, vitamins can include fat soluble vitamins such as vitamin A, vitamin D, vitamin E, and vitamin K and combinations thereof. In some embodiments, vitamins can include water soluble vitamins such as vitamin C (ascorbic acid), the B vitamins (thiamine or B₁, riboflavin or B₂, niacin or B₃, pyridoxine or B₆, folic acid or B₉, cyanocobalimin or B₁₂, pantothenic acid, biotin), and combinations thereof.

In some embodiments minerals can include but are not limited to sodium, magnesium, chromium, iodine, iron, manganese, calcium, copper, fluoride, potassium, phosphorous, molybdenum, selenium, zinc, and combinations thereof.

In some embodiments micronutrients can include but are not limited to L-carnitine, choline, coenzyme Q10, alpha-lipoic acid, omega-3-fatty acids, pepsin, phytase, trypsin, lipases, proteases, cellulases, and combinations thereof.

Antioxidants can include materials that scavenge free radicals. In some embodiments, antioxidants can include but are not limited to ascorbic acid, citric acid, rosemary oil, vitamin A, vitamin E, vitamin E phosphate, tocopherols, di-alpha-tocopheryl phosphate, tocotrienols, alpha lipoic acid, dihydrolipoic acid, xanthophylls, beta cryptoxanthin, lycopene, lutein, zeaxanthin, astaxanthin, beta-carotene, carotenes, mixed carotenoids, polyphenols, flavonoids, and combinations thereof.

In some embodiments phytochemicals can include but are not limited to cartotenoids, chlorophyll, chlorophyllin, fiber, flavonoids, anthocyanins, cyaniding, delphinidin, malvidin, pelargondin, peonidin, petunidin, flavanols, catechin, epicatechin, epigallocatechin, epigallocatechingallate (EGCG), theaflavins, thearubigins, proanthocyanins, flavonols, quercetin, kaempferol, myricetin, isorhamnetin, flavononeshesperetin, naringenin, eriodictyol, tangeretin, flavones, apigenin, luteolin, lignans, phytoestrogen, resveratrol, isoflavones, daidzein, genistein, glycitein, soy isoflavones, and combinations thereof.

### Mouth Moistening Ingredients

Mouth moisteners can include, but are not limited to, saliva stimulators such as acids and salts and combinations thereof. In some embodiments, acids can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof.

Mouth moisteners can also include hydrocolloid materials that hydrate and may adhere to oral surface to provide a sensation of mouth moistening. Hydrocolloid materials can include naturally occurring materials such as plant exudates, seed gums, and seaweed extracts or they can be chemically modified materials such as cellulose, starch, or natural gum derivatives. In some embodiments, hydrocolloid materials can include pectin, gum arabic, acacia gum, alginates, agar, carrageenans, guar gum, xanthan gum, locust bean gum, gelatin, gellan gum, cassia gum, galactomannans, tragacanth gum, karaya gum, curdlan, konjac, chitosan, xyloglucan, beta glucan, furcellaran, gum ghatti, tamarin, bacterial gums, and combinations thereof. Additionally, in some embodiments, modified natural gums such as propylene glycol alginate, carboxymethyl locust bean gum, low methoxyl pectin, and their combinations can be included. In some embodiments, modified celluloses can be included such as microcrystalline cellulose, carboxymethlcellulose (CMC), methylcellulose (MC), hydroxypropylmethylcellulose (HPCM), and hydroxypropylcellulose (MPC), and combinations thereof.

Similarly, humectants which can provide a perception of mouth hydration can be included. Such humectants can include, but are not limited to glycerol, sorbitol, polyethylene glycol, erythritol, and xylitol. Additionally, in some embodiments, fats can provide a perception of mouth moistening. Such fats can include medium chain triglycerides, vegetable oils, fish oils, mineral oils, and combinations thereof.

Mouth moisteners can also include pellitorine extracts, extracts of Anacyclus pyrethrum, trans-pellitorin, N-isobutyl-trans-2-trans-4-decadienamide, alkadienamides including N-isobutyl-E2, E4-decadienamide; N-isobutyl-E2, E4-undecadienamide; N-pyrollidyl-E2, E4-decadienamide; N-piperidyl-E2, E4-decadienamdie, or combinations thereof as disclosed in U.S. Patent Application No. 2007/0075368 which is incorporated in its entirety herein by reference; blends of n-isobutyldeca-trans-2-trans-4-dienamide with food acids as disclosed in U.S. Patent Application No. 2006/0204551 which is incorporated in its entirety herein by reference; blends of compounds according to formula (I): wherein R₁ represents C1-C2 n-alkyl; R₂ is 2-methyl-1-propyl and R₃ is hydrogen, or R₂ and R₃ taken together is a moiety having the formula—(CH₂)ₙ— wherein n is 4 or 5, or mixtures thereof with cooling compounds as disclosed in U.S. Patent Application No. 2007/0036838 to Keenan et al., which is incorporated in its entirety herein by reference.

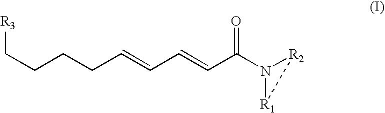

### Throat Care Ingredients

Throat soothing ingredients can include analgesics, anesthetics, demulcents, antiseptic, and combinations thereof. In some embodiments, analgesics/anesthetics can include menthol, phenol, hexylresorcinol, benzocaine, dyclonine hydrochloride, benzyl alcohol, salicyl alcohol, and combinations thereof. In some embodiments, demulcents can include but are not limited to slippery elm bark, pectin, gelatin, and combinations thereof. In some embodiments, antiseptic ingredients can include cetylpyridinium chloride, domiphen bromide, dequalinium chloride, and combinations thereof.

In some embodiments, antitussive ingredients such as chlophedianol hydrochloride, codeine, codeine phosphate, codeine sulfate, dextromethorphan, dextromethorphan hydrobromide, diphenhydramine citrate, and diphenhydramine hydrochloride, and combinations thereof can be included.

In some embodiments, throat soothing agents such as honey, propolis, aloe vera, glycerine, menthol and combinations thereof can be included. In still other embodiments, cough suppressants can be included. Such cough suppressants can fall into two groups: those that alter the consistency or production of phlegm such as mucolytics and expectorants; and those that suppress the coughing reflex such as codeine (narcotic cough suppressants), antihistamines, dextromethorphan and isoproterenol (non-narcotic cough suppressants). In some embodiments, ingredients from either or both groups can be included.

In still other embodiments, antitussives can include, but are not limited to, the group consisting of codeine, dextromethorphan, dextrorphan, diphenhydramine, hydrocodone, noscapine, oxycodone, pentoxyverine and combinations thereof. In some embodiments, antihistamines can include, but are not limited to, acrivastine, azatadine, brompheniramine, chlorpheniramine, clemastine, cyproheptadine, dexbrompheniramine, dimenhydrinate, diphenhydramine, doxylamine, hydroxyzine, meclizine, phenindamine, phenyltoloxamine, promethazine, pyrilamine, tripelennamine, triprolidine and combinations thereof. In some embodiments, non-sedating antihistamines can include, but are not limited to, astemizole, cetirizine, ebastine, fexofenadine, loratidine, terfenadine, and combinations thereof.

In some embodiments, expectorants can include, but are not limited to, ammonium chloride, guaifenesin, ipecac fluid extract, potassium iodide and combinations thereof. In some embodiments, mucolytics can include, but are not limited to, acetylcysteine, ambroxol, bromhexine and combinations thereof. In some embodiments, analgesic, antipyretic and anti-inflammatory agents can include, but are not limited to, acetaminophen, aspirin, diclofenac, diflunisal, etodolac, fenoprofen, flurbiprofen, ibuprofen, ketoprofen, ketorolac, nabumetone, naproxen, piroxicam, caffeine and mixtures thereof. In some embodiments, local anesthetics can include, but are not limited to, lidocaine, benzocaine, phenol, dyclonine, benzonotate and mixtures thereof.

In some embodiments nasal decongestants and ingredients that provide the perception of nasal clearing can be included. In some embodiments, nasal decongestants can include but are not limited to phenylpropanolamine, pseudoephedrine, ephedrine, phenylephrine, oxymetazoline, and combinations thereof. In some embodiments ingredients that provide a perception of nasal clearing can include but are not limited to menthol, camphor, borneol, ephedrine, eucalyptus oil, peppermint oil, methyl salicylate, bornyl acetate, lavender oil, wasabi extracts, horseradish extracts, and combinations thereof. In some embodiments, a perception of nasal clearing can be provided by odoriferous essential oils, extracts from woods, confectioneries, flowers and other botanicals, resins, animal secretions, and synthetic aromatic materials.

The features and advantages of the present invention are more fully shown by the following examples which are provided for purposes of illustration, and are not to be construed as limiting the invention in any way.

### EXAMPLES

### Example 1

### Oral Composition

The oral composition is prepared by combining the components as set forth in Examples A-P in Tables 1 and 2. The amounts are based on the weight percent of the total oral composition.

TABLE 1
Oral Compositions

| | % by weight | | | | | | | |
|---|---|---|---|---|---|---|---|---|
| Component | A | B | C | D | E | F | G | H |
| 4-pentenyl isothiocyanate | 100 | | | | 15-85 | | 15-85 | 15-85 |
| 5-hexenyl isothiocyanate | | 100 | | | 15-85 | 15-85 | 15-85 | 15-85 |
| 3-butenyl isothiocyanate | | | 100 | | | | 15-85 | 15-85 |
| 3-methylthiopropyl isothiocyanate | | | | 100 | | 15-85 | | 15-85 |

The oral composition for each Example is prepared by combining the ingredients in the amounts indicated in Table 1. The ingredients would be added sequentially according to the formulation and mixed at ambient temperature until homogeneous.

TABLE 2
Oral Compositions

| | % by weight | | | | | | | |
|---|---|---|---|---|---|---|---|---|
| Component | I | J | K | L | M | N | O | P |
| 4-pentenyl | 50 | | | | 40 | | 30 | 20 |
| isothiocyanate | | | | | | | | |
| 5-hexenyl | | 75 | | | 40 | 25 | 30 | 20 |
| isothiocyanate | | | | | | | | |
| 3-butenyl | | | 80 | | | | 30 | 20 |
| isothiocyanate | | | | | | | | |
| 3-methylthiopropyl | | | | 80 | | 25 | | 20 |
| isothiocyanate | | | | | | | | |
| Cooling agent | 50 | | | | | 50 | | |
| Warming agent | | 25 | | | | | 10 | |
| Tingling agent | | | 20 | | | | | |
| Sweetness | | | | 20 | | | | 20 |
| enhancer | | | | | | | | |
| Umami enhancer | | | | | 20 | | | |

The oral composition for each Example is prepared by combining the ingredients in the amounts indicated in Table 2. The ingredients would be added sequentially according to the formulation and mixed at ambient temperature until homogeneous. Alternatively, the ingredients are added sequentially according to the formulation and mixed with heating to facilitate dissolution.

### Example 2

### Center-Fill Confectionery Composition

The center-fill composition is prepared by combining the components as set forth in Examples I-L in Table 3. The amounts included are based on the weight percent of the total center-fill composition.

TABLE 3
Center-fill Composition

| | % by weight | | | |
|---|---|---|---|---|
| Component | I | J | K | L |
| Sugar | 42-48 | 42-48 | | |
| Corn Syrup | 42-48 | 42-48 | | |
| Polyol (syrup | | | 84-96 | 84-96 |
| or slurry) | | | | |
| Guar Gum | 0.1-0.7 | | 0.1-0.7 | |
| Citric Acid | 0.7-4.5 | 0.7-4.5 | 0.7-4.5 | 0.7-4.5 |
| Flavor | 0.05-.30 | 0.05-.30 | 0.05-.30 | 0.05-.30 |
| Color | 0.1-0.7 | 0.1-0.7 | 0.1-0.7 | 0.1-0.7 |
| 4-pentenyl | 0.005-10 | | 0.005-10 | |
| isothiocyanate | | | | |
| 5-hexenyl | 0.005-10 | | 0.005-10 | |
| isothiocyanate | | | | |
| 3-butenyl | | 0.005-10 | | 0.005-10 |
| isothiocyanate | | | | |
| 3- | | 0.005-10 | | 0.005-10 |
| methyl- | | | | |
| thiopropyl | | | | |
| isothiocyanate | | | | |

The confectionery composition is prepared by combining the components as set forth in Examples U-II in Tables 4-6.

The gummy candy shell composition is prepared by combining the components as set forth in Examples U-Z in Table 4. The amounts included are based on the weight percent of the total gummy candy shell composition.

TABLE 4
Gummy Candy Shell Composition

| | % by weight | | | | | |
|---|---|---|---|---|---|---|
| Component | U | V | W | X | Y | Z |
| Sugar | 15-25 | 15-25 | | | 20-35 | |
| Corn Syrup | 30-45 | 30-45 | | | 20-35 | |
| Xylitol | | | 45-70 | | | |
| Erythritol | | | | 45-70 | | 20-35 |
| HSH | | | | | | 20-35 |
| 4-pentenyl | 0.001-3 | | | 0.001-3 | | |
| isothiocyanate | | | | | | |
| 5-hexenyl | | 0.001-3 | | | 0.001-3 | |
| isothiocyanate | | | | | | |
| 3-butenyl | | | 0.001-3 | | | 0.001-3 |
| isothiocyanate | | | | | | |
| 3- | | | | 0.001-3 | 0.001-3 | 0.001-3 |
| methylthiopropyl | | | | | | |
| isothiocyanate | | | | | | |
| Gelatin | 7-12 | | 7-12 | | | 7-12 |
| Pectin | 0.8-1.5 | | 0.8-1.5 | | | 0.8-1.5 |
| Carrageenan | | 1.5-3.0 | | 1.5-3.0 | 1.5-3.0 | |
| Color | 0.1-0.7 | 0.1-0.7 | 0.1-0.7 | 0.1-0.7 | 0.1-0.7 | 0.1-0.7 |
| Flavor | 0.05-0.30 | 0.05-0.30 | 0.05-0.30 | 0.05-0.30 | 0.05-0.30 | 0.05-0.30 |

The hard candy shell composition is prepared by combining the components as set forth in Examples AA-DD in Table 5. The amounts included are based on the weight percent of the total hard candy shell composition.

TABLE 5
Hard Candy Shell Composition

| | % by weight | | | |
|---|---|---|---|---|
| Component | AA | BB | CC | DD |
| Sugar | 15-25 | 15-25 | 20-35 | 20-35 |
| Corn Syrup | 30-45 | 30-45 | 20-35 | 20-35 |
| 4-pentenyl | 0.001-3 | | | |
| isothiocyanate | | | | |
| 5-hexenyl | | 0.001-3 | 0.001-3 | |
| isothiocyanate | | | | |
| 3-butenyl | | | | 0.001-3 |
| isothiocyanate | | | | |
| 3- | | | 0.001-3 | 0.001-3 |
| methyl- | | | | |
| thiopropyl | | | | |
| isothiocyanate | | | | |
| Pectin | 0.8-1.5 | | | 0.8-1.5 |
| Carrageenan | | 1.5-3.0 | 1.5-3.0 | |
| Color | 0.1-0.7 | 0.1-0.7 | 0.1-0.7 | 0.1-0.7 |
| Flavor | 0.05-0.30 | 0.05-0.30 | 0.05-0.30 | 0.05-0.30 |

The sugar-free hard candy shell composition is prepared by combining the components as set forth in Examples EE-II in Table 6. The amounts included are based on the weight percent of the total sugar-free hard candy shell composition.

TABLE 6
Sugar-Free Hard Candy Shell Composition

| | % by weight | | | | |
|---|---|---|---|---|---|
| Component | EE | FF | GG | HH | II |
| Xylitol | 45-70 | | | | |
| Erythritol | | 45-70 | 20-35 | | |
| Isomalt | | | | 45-70 | |
| Maltitol | | | | | 45-70 |
| HSH | | | 20-35 | | |
| 4-pentenyl | | 0.001-3 | | 0.001-3 | |
| isothiocyanate | | | | | |
| 5-hexenyl | 0.001-3 | | | | |
| isothiocyanate | | | | | |
| 3-butenyl | 0.001-3 | | 0.001-3 | | 0.001-3 |
| isothiocyanate | | | | | |
| 3- | | 0.001-3 | 0.001-3 | | |
| methylthiopropyl | | | | | |
| isothiocyanate | | | | | |
| Pectin | 0.8-1.5 | | 0.8-1.5 | | 0.8-1.5 |
| Carrageenan | | 1.5-3.0 | | 1.5-3.0 | |
| Color | 0.1-0.7 | 0.1-0.7 | 0.1-0.7 | 0.1-0.7 | 0.1-0.7 |
| Flavor | 0.05-0.30 | 0.05-0.30 | 0.05-0.30 | | |

The optional coating composition is prepared by combining the components as set forth in Examples JJ-NN in Table 7. The amounts included are based on the weight percent of the total coating composition.

TABLE 7
Particulate Coating Composition

| | % by weight | | | | |
|---|---|---|---|---|---|
| Component | JJ | KK | LL | MM | NN |
| Particulate Sugar | 68-99.999 | 68-99.999 | | | 68-99.999 |
| Particulate Polyol | | | 68-99.999 | 68-99.999 | |
| Citric Acid | 0-10 | 0-10 | 0-10 | 0-10 | 0-10 |
| Malic Acid | 0-10 | 0-10 | 0-10 | 0-10 | 0-10 |
| Lactic Acid | 0-10 | 0-10 | 0-10 | 0-10 | 0-10 |
| 4-pentenyl | | | 0.001-1 | 0.001-1 | |
| isothiocyanate | | | | | |
| 5-hexenyl | 0.001-1 | | 0.001-1 | 0.001-1 | |
| isothiocyanate | | | | | |
| 3-butenyl | | 0.001-1 | 0.001-1 | | |
| isothiocyanate | | | | | |
| 3-methylthiopropyl | | 0.001-1 | | 0.001-1 | 0.001-1 |
| isothiocyanate | | | | | |

Any of the center fill compositions of Examples Q-T are incorporated into any of the gummy candy shell compositions of Examples U-Z, hard candy shell compositions of Examples AA-DD or sugar-free hard candy shell compositions Examples EE-II and then optionally, any of the coating compositions of Examples JJ-NN are applied to the exterior. The center fill is added in an amount from about 5% by weight to about 25% by weight of the total composition. The gummy candy shell or hard candy shell is added in an amount from about 70% by weight to about 90% by weight of the total composition and the coating is added in an amount from about 5% by weight to about 15% by weight of the total composition. The isothiocyanates can be incorporated into the particulate coating as particulates or they can be added as liquids that are plated or absorbed onto the particulate sugar(s) and/or polyol(s).

### Example 3

### Chewing Gum Composition

The chewing gum composition is prepared by combining the components as set forth in Examples OO-SS in Table 8. The amounts are based on the weight percent of the chewing gum composition.

TABLE 8
Chewing Gum Composition Containing Isothiocyanate

| | % by weight | | | | |
|---|---|---|---|---|---|
| Component | OO | PP | QQ | RR | SS |
| Chewing Gum | 39.00 | 39.00 | 39.00 | 39.00 | 39.00 |
| Base | | | | | |
| Sorbitol | QS | QS | QS | QS | QS |
| Mannitol | 9.00 | 9.00 | 9.00 | 9.00 | 9.00 |
| Flavor | 3.67 | 3.67 | 3.67 | 3.67 | 3.67 |
| Glycerin | 1.50 | 1.50 | 1.50 | 1.50 | 1.50 |
| Lecithin | 0.20 | 0.20 | 0.20 | 0.20 | 0.20 |
| Aspartame | 0.30 | 0.30 | 0.30 | 0.30 | 0.30 |
| AceK | 0.15 | 0.15 | 0.15 | 0.15 | 0.15 |
| Encapsulated | 6.00 | 6.00 | 6.00 | 6.00 | 6.00 |
| Xylitol | | | | | |
| 4-pentenyl | 0.0050-10 | 0.00-10 | 0.00-10 | 0.0050-10 | 0.00-10 |
| isothiocyanate | | | | | |
| 5-hexenyl | 0.00-10 | 0.0050-10 | 0.00-10 | 0.00-10 | 0.0050-10 |
| isothiocyanate | | | | | |
| 3-butenyl | 0.00-10 | 0.00-10 | 0.0050-10 | 0.00-10 | 0.0050-10 |
| isothiocyanate | | | | | |
| 3-methylthiopropyl | 0.00-10 | 0.00-10 | 0.00-10 | 0.0050-10 | 0.00-10 |
| isothiocyanate | | | | | |

A chewing gum composition is prepared according to the composition in Table 8 above.

The chewing gum base is melted in a mixer. The remaining ingredients are added to the molten chewing gum base. The melted chewing gum base with ingredients is mixed to completely disperse the ingredients. The resulting chewing gum is allowed to cool. The cooled chewing gum is sized and conditioned for about a week and packaged.

The resulting mix then is then formed into the desired final shape employing conventional techniques, e.g., extruded, rolled and cut into sticks, cast into pellets and then optionally coated, or pressed into tablets, among others.

### Example 4

### Center-Fill Chewing Gum Composition

The chewing gum composition is prepared by combining the components as set forth in Examples TT-QQQ in Tables 9-11.

The chewing gum region composition is prepared by combining the components as set forth in Examples TT-AAA in Table 6. The amounts included are based on the weight percent of the total chewing gum region composition.

TABLE 6
Chewing gum Region Composition

| | % by weight | | | | | | | |
|---|---|---|---|---|---|---|---|---|
| Component | TT | UU | VV | WW | XX | YY | ZZ | AAA |
| Chewing gum | 28-42 | 28-42 | 28-42 | 28-42 | 28-42 | 28-42 | 28-42 | 28-42 |
| base* | | | | | | | | |
| Lecithin | 0.25 | 0.25 | 0.05 | 0.05 | 0.05 | 0.05 | 0.05 | 0.05 |
| Maltitol | 52-55 | 45-50 | 46-50 | 50-54 | 52-57 | 45-55 | 47-52 | 50-55 |
| Sorbitol | 0 | 0-10 | 5-10 | 0-5 | 0-5 | 5-10 | 0-5 | 0-5 |
| Lycasin ™ | 0 | 0 | 0.25 | 0.25 | 0.25 | 0.25 | 0.25 | 0.25 |
| Flavors | 2.50 | 2.50 | 2.26 | 2.26 | 2.26 | 2.50 | 2.50 | 2.50 |
| Cooling agent | 0.08 | 0.08 | 0 | 0 | 0 | 0.08 | 0.08 | 0.08 |
| Acidulants | 1.2 | 1.2 | 0 | 0 | 0 | 1.2 | 1.2 | 1.2 |
| Intense sweetener | 3.40 | 3.40 | 3.40 | 3.40 | 3.40 | 3.40 | 3.40 | 3.40 |
| 4-pentenyl | 0.005-10 | 0-10 | 0-10 | 0.005-10 | 0.005-10 | 0-10 | 0-10 | 0.005-10 |
| isothiocyanate | | | | | | | | |
| 5-hexenyl | 0-10 | 0.005-10 | 0-10 | 0.005-10 | 0-10 | 0.005-10 | 0-10 | 0.005-10 |
| isothiocyanate | | | | | | | | |
| 3-butenyl | 0-10 | 0-10 | 0.005-10 | 0.005-10 | 0-10 | 0-10 | 0.005-10 | 0-10 |
| isothiocyanate | | | | | | | | |
| 3-methylthiopropyl | 0-10 | 0-10 | 0-10 | 0.005-10 | 0.005-10 | 0.005-10 | 0.005-10 | 0-10 |
| isothiocyanate | | | | | | | | |

*chewing gum base may include 3% to 11% by weight of a filler such as, for example, talc, dicalcium phosphate, and calcium carbonate (the amount of filler in the chewing gum base is based on the weight percent of the chewing gum region composition, for example, in the above compositions A-H, if a chewing gum region composition includes 5% filler, the amount of chewing gum base will be 5% less than the range recited in the table, i.e., from 23-37%)

The liquid-fill composition is prepared by combining the components as set forth in Examples BBB-III in Table 10. The amounts included are based on the weight percent of the total liquid-fill composition.

TABLE 10
Liquid-fill Composition

| | % by weight | | | | | | | |
|---|---|---|---|---|---|---|---|---|
| Component | BBB | CCC | DDD | EEE | FFF | GGG | HHH | III |
| Glycerin | 63.00 | 63.00 | 63.00 | 30.00 | 63.00 | 63.00 | 1.50 | 63.00 |
| Lycasin ™ | 29.26 | 29.26 | 29.49 | 56.00 | 29.49 | 29.17 | 65.22 | 29.17 |
| Sorbitol solution | 3.25 | 3.25 | 3.28 | 7.50 | 3.28 | 3.24 | 28.60 | 3.24 |
| Sodium carboxymethyl | 0.08 | 0.008 | 0.15 | 0.25 | 0.15 | 0.20 | 0.20 | 0.20 |
| cellulose | | | | | | | | |
| Color | 0.004 | 0.004 | 0.0004 | 0.004 | 0.0004 | 0.004 | 0.004 | 0.004 |
| Flavors | 1.30 | 1.30 | 4.00 | 4.00 | 4.00 | 0.30 | 1.40 | 0.30 |
| Cooling agent | 0.06 | 0.06 | 0.06 | 0.06 | 0.06 | 0.06 | 0.06 | 0.06 |
| Citric acid | 3.00 | 3.00 | 0 | 2.17 | 0 | 3.00 | 3.00 | 3.00 |
| Intense sweetener | 0.05 | 0.05 | 0.02 | 0.02 | 0.02 | 0.02 | 0.02 | 0.02 |
| 4-pentenyl | 0.05-10 | 0-10 | 0-10 | 0-10 | 0.05-10 | 0-10 | 0-10 | 0-10 |
| isothiocyanate | | | | | | | | |
| 5-hexenyl isothiocyanate | 0-10 | 0.05-10 | 0-10 | 0-10 | 0-10 | 0.05-10 | 0-10 | 0-10 |
| 3-butenyl isothiocyanate | 0-10 | 0-10 | 0.05-10 | 0-10 | 0-10 | 0-10 | 0.05-10 | 0-10 |
| 3-methylthiopropyl | 0-10 | 0-10 | 0-10 | 0.05-10 | 0.05-10 | 0.05-10 | 0.05-10 | 0-10 |
| isothiocyanate | | | | | | | | |

The optional coating composition is prepared by combining the components as set forth in Examples JJJ-QQQ in Table 11. The amounts included are based on the weight percent of the total coating composition.

TABLE 11
Coating Composition

| | % by weight | | | | | | | |
|---|---|---|---|---|---|---|---|---|
| Component | JJJ | KKK | LLL | MMM | NNN | OOO | PPP | QQQ |
| Maltitol | 95 | 95 | | | | | | 95 |
| Isomalt | | | | 95 | | 95 | 95 | |
| Sucrose | | | 95 | | 95 | | | |
| Bleached gum Arabic | 3 | 3 | 3 | 3 | 3 | 3 | 3 | 3 |
| Titanium dioxide | 0.3 | 0.3 | 0.3 | 0.3 | 0.3 | 0.3 | 0.3 | 0.3 |
| Flavors | 1 | 1 | 0.5 | 0.5 | 0.5 | 1 | 1 | 1 |
| 4-pentenyl | 0.001-3 | | | 0.001-3 | 0.001-3 | | | 0.001-3 |
| isothiocyanate | | | | | | | | |
| 5-hexenyl | | 0.001-3 | 0.001-3 | 0.001-3 | | 0.001-3 | 0.001-3 | 0.001-3 |
| isothiocyanate | | | | | | | | |
| 3-butenyl isothiocyanate | | 0.001-3 | 0.001-3 | 0.001-3 | | 0.001-3 | 0.001-3 | 0.001-3 |
| 3-methylthiopropyl | | | 0.001-3 | 0.001-3 | | | 0.001-3 | 0.001-3 |
| isothiocyanate | | | | | | | | |

Chewing gum pieces including three regions: liquid fill, chewing gum region and coating are prepared according to the compositions in Tables 9-11 above with each region according to the corresponding components for compositions TT-QQQ.

The compositions for the chewing gum regions are prepared by first combining talc, where present, with the chewing gum base under heat at about 85° C. This combination is then mixed with the maltitol, lecithin and other polyols for six minutes. The flavor blends which include a pre-mix of the flavors, cooling agents and isothiocyanates are added and mixed for 1 minute. Finally, the acids and intense sweeteners are added and mixed for 5 minutes.

The liquid fill composition is then prepared by first preparing a pre-mix of the sodium carboxymethyl cellulose, glycerine, and polyols. This pre-mix is then combined with the colors, flavors, cooling agents, acids, intense sweeteners and isothiocyanates and mixed.

The chewing gum region and liquid-fill compositions are then extruded together and formed into tablets by the process described above at paragraphs [0112]-[0116 ] above. The chewing gum pieces each have a total weight of approximately 2.2 g. In the final chewing gum pieces, the chewing gum region is about 62% by weight, the liquid-fill is about 8% by weight, and the coating is about 30% by weight.

The coating composition can be applied to the liquid-filled chewing gum compositions by any known means including hard panning, soft panning, melt spraying, and the like.

### Example 5

### Hard Candy Composition

The hard candy composition is prepared by combining the components as set forth in Table 9. The amounts included are based on the weight percent of the total hard candy composition.

TABLE 9
Hard Boiled Candy with Isothiocyanate

| Component | % by weight |
|---|---|
| Candy Base (sugar, glucose syrup 42DE and water) | 85.05 |
| Color solution | 0.40 |
| Pectin solution (8% pectin powder, potassium | 13.75 |
| citrate, water) | |
| Flavor | 0.19 |
| 4-pentenyl isothiocyanate | 0.001-3.0 |
| 5-hexenyl isothiocyanate | 0.001-3.0 |
| 3-butenyl isothiocyanate | 0.001-3.0 |
| 3-methylthiopropyl isothiocyanate | 0.001-3.0 |

A hard candy is prepared according to the formulation in Table 9 above.

Pectin and potassium citrates are dispersed in hot water (70° C.) with vigorous mixing to prepare the pectin solution. The candy composition is prepared by dissolving the sugar/glucose syrup in water and cooking at 146° C. The pectin solution, color and isothiocyanates are added to the cooked sugar mass. The batch is placed on a cooling table where flavor, warming agent and acids are added. The batch is kneaded. Roll candies are dropped into the desired weight and size pieces.

The hard candy composition is prepared by combining the components as set forth in Examples RRR-UUU in Table 13. The amounts included are based on the weight percent of the total hard candy composition.

TABLE 13
Hard Candy Composition

| | % by weight | | | |
|---|---|---|---|---|
| Component | RRR | SSS | TTT | UUU |
| Sugar | 15-25 | 15-25 | 20-35 | 20-35 |
| Corn Syrup | 30-45 | 30-45 | 20-35 | 20-35 |
| 4-pentenyl | 0.001-3 | | | |
| isothiocyanate | | | | |
| 5-hexenyl | | 0.001-3 | 0.001-3 | |
| isothiocyanate | | | | |
| 3-butenyl | | | | 0.001-3 |
| isothiocyanate | | | | |
| 3- | | | 0.001-3 | 0.001-3 |
| methyl- | | | | |
| thiopropyl | | | | |
| isothiocyanate | | | | |
| Pectin | 0.8-1.5 | | | 0.8-1.5 |
| Carrageenan | | 1.5-3.0 | 1.5-3.0 | |
| Color | 0.1-0.7 | 0.1-0.7 | 0.1-0.7 | 0.1-0.7 |
| Flavor | 0.05-0.30 | 0.05-0.30 | 0.05-0.30 | 0.05-0.30 |

The sugar-free hard candy shell composition is prepared by combining the components as set forth in Examples VVV-ZZZ in Table 14. The amounts included are based on the weight percent of the total sugar-free hard candy shell composition.

TABLE 14
Sugar-Free Hard Candy Shell Composition

| | % by weight | | | | |
|---|---|---|---|---|---|
| Component | VVV | WWW | XXX | YYY | ZZZ |
| Xylitol | 45-70 | | | | |
| Erythritol | | 45-70 | 20-35 | | |
| Isomalt | | | | 45-70 | |
| Maltitol | | | | | 45-70 |
| HSH | | | 20-35 | | |
| 4-pentenyl | | 0.001-3 | | 0.001-3 | |
| isothiocyanate | | | | | |
| 5-hexenyl | 0.001-3 | | | | |
| isothiocyanate | | | | | |
| 3-butenyl | 0.001-3 | | 0.001-3 | | 0.001-3 |
| isothiocyanate | | | | | |
| 3- | | 0.001-3 | 0.001-3 | | |
| methylthiopropyl | | | | | |
| isothiocyanate | | | | | |
| Pectin | 0.8-1.5 | | 0.8-1.5 | | 0.8-1.5 |
| Carrageenan | | 1.5-3.0 | | 1.5-3.0 | |
| Color | 0.1-0.7 | 0.1-0.7 | 0.1-0.7 | 0.1-0.7 | 0.1-0.7 |
| Flavor | 0.05-0.30 | 0.05-0.30 | 0.05-0.30 | | |

1. An oral composition comprising a wasabi extract comprising:
a compound selected from the group consisting of 4-pentenyl isothiocyanate, 5-hexenyl isothiocyanate, 3-butenyl isothiocyanate, 3-methylthiopropyl isothiocyanate, and combinations thereof.

2. The oral composition of claim 1, further comprising at least one sensate.

3. The oral composition of claim 1, further comprising at least one potentiator.

4. A composition comprising:
(a) a confectionery carrier; and
(b) a compound selected from the group consisting of substituted or unsubstituted C₄₋₁₀ alkenyl isothiocyanates, 3-methylthiopropyl isothiocyanate, and combinations thereof.

5. The confectionery composition of claim 4, wherein said compound is selected from the group consisting of 4-pentenyl isothiocyanate, 5-hexenyl isothiocyanate, 3-butenyl isothiocyanate, 3-methylthiopropyl isothiocyanate, and combinations thereof.

6. The confectionery composition of claim 4, further comprising a coating surrounding said confectionery composition.

7. The confectionery composition of claim 6, wherein said coating comprises at least one compound selected from the group consisting of 4-pentenyl isothiocyanate, 5-hexenyl isothiocyanate, 3-butenyl isothiocyanate, 3-methylthiopropyl isothiocyanate, and combinations thereof.

8. The confectionery composition of claim 4, wherein said confectionery carrier is selected from the group consisting of hard candy, soft candy, chewy candy, center-fill candy, multi-layer candy, cotton candy, pressed tablets, multi-layer pressed tablets, lozenges, edible films, nougats, caramels, frappes, granules, gummies, jellies, taffies, toffees, and chewing gum.

9. The confectionery composition of claim 8, wherein said chewing gum is selected from the group consisting of slab, pellet, sticks, balls, cubes, center-fill chewing gums, candy chewing gums, multi-layer chewing gums, deposited chewing gums and compressed chewing gums.

10. The confectionery composition of claim 4, wherein said confectionery composition comprises a multi-region confectionery composition comprising:
(i) a first region;
(ii) a second confectionery shell region surrounding said first region, said confectionery shell region comprising said confectionery carrier; and
(iii) said compound selected from the group consisting of substituted or unsubstituted C₄₋₁₀ alkenyl isothiocyanates, 3-methylthiopropyl isothiocyanate, and combinations thereof being located in one or more of said first region and said second confectionery shell region.

11. The confectionery composition of claim 10, further comprising a third region surrounding said confectionery composition.

12. The confectionery composition of claim 11, wherein said third region comprises at least one compound selected from the group consisting of 4-pentenyl isothiocyanate, 5-hexenyl isothiocyanate, 3-butenyl isothiocyanate, 3-methylthiopropyl isothiocyanate, and combinations thereof is located in said third region.

13. A method of preparing a confectionery product, comprising the steps of:
(a) providing at least one sweetener;
(b) adding a compound selected from the group consisting of 4-pentenyl isothiocyanate, 5-hexenyl isothiocyanate, 3-butenyl isothiocyanate, 3-methylthiopropyl isothiocyanate, and combinations thereof to said at least one sweetener; and
(c) forming a confectionery product from the combination of (a) and (b).

14. The method of claim 13, wherein said step of forming a confectionery product comprises the steps of shaping the combination of (a) and (b) and cutting the shaped combination into a desired piece.

15. The method of claim 13, wherein said step of forming a confectionery product comprises the step of depositing the combination of (a) and (b) into a mold to form a confectionery product.

16. A method of preparing a chewing gum product, comprising the steps of:
(a) providing an elastomer;
(b) adding a compound selected from the group consisting of 4-pentenyl isothiocyanate, 5-hexenyl isothiocyanate, 3-butenyl isothiocyanate, 3-methylthiopropyl isothiocyanate, and combinations thereof to said elastomer; and
(c) forming a chewing gum product from the combination of (a) and (b).

17. The method of claim 16, wherein said step of forming a chewing gum product comprises the steps of shaping the combination of (a) and (b) and cutting the shaped combination into a desired piece.

18. The method of claim 16, wherein said step of forming a chewing gum product comprises the step of depositing the combination of (a) and (b) into a mold to form a chewing gum product.

19. A method of preparing a multi-region confectionery composition comprising the steps of:
(a) extruding a multi-region rope of a confectionery layer, wherein said rope comprises:
(i) a first region composition;
(ii) a second confectionery shell region surrounding said first region composition; and
(iii) a compound selected from the group consisting of 4-pentenyl isothiocyanate, 5-hexenyl isothiocyanate, 3-butenyl isothiocyanate, 3-methylthiopropyl isothiocyanate, and combinations thereof, wherein said compound is located in one or more of said first region composition and said second confectionery shell region;
(b) sizing said rope;
(c) feeding said rope into a forming mechanism; and
(d) forming individual pieces of said multi-region rope.

20. The method of claim 19, further comprising the step of applying a coating to said pieces of said multi-region rope.

21. The method of claim 20, further comprising the step of co-extruding said multi-region rope with a coating layer prior to said step of sizing said rope such that said coating layer at least partially surrounds said multi-region rope.

22. A method of imparting the perception of nasal clearing to a user comprising the steps of:
(a) providing an oral delivery system comprising a confectionery composition comprising a compound selected from the group consisting of 4-pentenyl isothiocyanate, 5-hexenyl isothiocyanate, 3-butenyl isothiocyanate, 3-methylthiopropyl isothiocyanate, and combinations thereof; and
(b) orally administering said oral delivery system for a time sufficient to produce a nasal clearing perception.

23. A method of preparing a flavor composition comprising:
(a) combining at least one flavor compound with a compound selected from the group consisting of 4-pentenyl isothiocyanate, 5-hexenyl isothiocyanate, 3-butenyl isothiocyanate, 3-methylthiopropyl isothiocyanate, and combinations thereof; and
(b) mixing until homogeneous.

24. The method of claim 23, further comprising encapsulating said flavor composition.

25. The method of claim 23, further comprising adding at least one sensate to form a flavor sensate blend.

26. The method of claim 25, further comprising encapsulating said flavor sensate blend.

27. The method of claim 25, further comprising heating said flavor sensate blend to dissolve said at least one sensate.

28. The method of claim 25, further comprising encapsulating said flavor sensate blend.
